(19) United States
(12) Patent Application Publication
(10) Pub. No.: US 2021/0145838 A1
(43) Pub. Date: May 20, 2021

(54) SELECTIVE PDE4D INHIBITORS AGAINST DEMYELINATING DISEASES

(71) Applicants: Universiteit Hasselt, Hasselt (BE); Universiteit Maastricht, Maastricht (NL); Academisch Ziekenhuis Maastricht, Maastricht (NL)

(72) Inventors: Tim Vanmierlo, Bocholt (BE); Jos Prickaerts, Maastricht (NL); Paul Wieringa, Maastricht (NL)

(21) Appl. No.: 17/044,894

(22) PCT Filed: Apr. 4, 2019
(86) PCT No.: PCT/EP2019/058495

§ 371 Date: Oct. 2, 2020

(30) Foreign Application Priority Data
Apr. 5, 2018 (EP) 18165843.6

(51) Int. Cl. A61K 31/5377; A61K 31/505; A61P 25/28

(52) U.S. Cl. CPC A61K 31/5377; A61P 25/28; A61K 31/505

(57) ABSTRACT

The current invention relates to selective PDE4D inhibitors for use in the prevention and/or treatment of demyelinating diseases of the central nervous system and of the peripheral nervous system.

### FIELD OF THE INVENTION

The current invention relates to selective PDE4D inhibitors for use in the prevention and/or treatment of demyelinating diseases of the central nervous system and of the peripheral nervous system, such as for example multiple sclerosis, neuropathy or traumatic nerve injury.

### BACKGROUND TO THE INVENTION

Demyelinating diseases are a group of neurological disorders in which myelin, the substance surrounding axons of neurons, degenerates. As a result, the axon's ability to conduct electrical signals degenerates. The most common demyelinating disease is multiple sclerosis, a demyelinating disorder of the central nervous system (CNS). Demyelinating diseases can also be related to the peripheral nervous system, such as for example different types of neuropathy, Marie-Charcot tooth disease or traumatic nerve injury.

Multiple sclerosis (MS) is characterized by a variety of clinical symptoms, such as gradual muscle weakness, fatigue, and cognitive impairment. The destructive immunological interplay at disease onset leads to oligodendrocyte loss, focal demyelination, and axonal damage. Available therapies modulate the immune response to temper early disease activity, but have limited efficacy in preventing the transition towards the chronic stage and are no longer effective in the progressive stage of MS (pMS). Hence, there is an urgent need for therapies that halt disease progression and boost repair processes.

Early in the course of Multiple Sclerosis (MS), neuroinflammation not only induces demyelination but at the same time activates endogenous repair mechanisms (remyelination). Early remyelination is characterized by the expansion and mobilization of oligodendrocyte progenitor cells (OPCs). OPCs rapidly remyelinate affected axons, yielding remyelinated shadow plaques in the Central Nerve System (CNS). Despite the presence of sufficient numbers of OPCs in the vicinity of the pathological lesions, endogenous repair mechanisms frequently fail in pMS, resulting in chronically demyelinated axons embedded in gliotic scar tissue. This has profound pathophysiological consequences. Loss of myelin not only disrupts axonal function per se, but it also compromises the physical integrity of axons by increasing susceptibility to inflammatory mediators and disrupting trophic support provided by myelinating oligodendrocytes.

The processes underlying impaired endogenous repair mechanisms are poorly understood, but there is now strong evidence that this is related to the inability of OPCs to differentiate into myelin-forming oligodendrocytes. Identification of factors that relieve the block in OPC differentiation will allow us to restore endogenous remyelination, a strategy predicted to limit disease progression in pMS and significantly improve disability.

Phosphodiesterases (PDEs) are a class of enzymes that hydrolyze and inactivate cyclic oligonucleotides (cAMP and cGMP). Cyclic oligonucleotides are second messengers that translate an extracellular signal such as a growth factor binding to its receptor into cellular differentiation. PDEs have been classified in 11 families (PDE1-11) based on subcellular distribution, mechanisms of regulation, and enzymatic and kinetic properties. Most of these families consist of several gene products (e.g. PDE4A-4D), yielding a cell type-specific PDE expression signature.

Previously, it has been shown that aspecific inhibition of PDE4 supports OPC differentiation and neuronal survival in a model of spinal cord injury, reduces neuroinflammation in an animal model for MS, and improves neuroplasticity and cognitive parameters such as learning and memory in different species.

The genes PDE4A and PDE4B are known to show a higher expression level in oligodendrocytes, compared to the PDE4D gene, which shows a 10-fold lower expression (Zhang et al., 2014). The pan-PDE4 inhibitor roflumilast, which inhibits all PDE4 isoforms, induces in vitro and in vivo remyelination as well as an improved cognitive behavior. Yet, despite these neuroprotective features, the use of pan-PDE4 inhibitors coincides with emetic side effects (e.g. nausea) at the repair-inducing dose. It is further known that inhibition of isoforms of PDE4D and PDE4B may induce emesis (Bruno et al., 2011; Giembycz et al., 2002), and administration of anti-emetic substances seems to be insufficient to circumvent these side-effects, rendering these compounds unsuitable for clinical use.

We surprisingly, we found that selective PDE4D inhibitors, such as for example Gebr32a and BPN14770, boosted OPC differentiation in primary OPCs in vitro. Comparably, we confirmed that selective PDE4D inhibition improved (re)myelination in ex vivo demyelinated cerebellar brain slices.

In addition, a skilled person may expect that higher doses of selective PDE4D inhibitors are needed to induce remyelination when compared to roflumilast, which already displayed emetic side-effects at the remyelination-inducing dose. After all, PDE4 enzymes contribute equally to the total concentration of cAMP, which is necessary for remyelination. As the concentration of cAMP is generated in the brains by the common involvement of PDE4A, PDE4B and PDE4D (PDE4C is not present in the brains), one should expect that the active concentration of a PDE4D-specific inhibitor (e.g. Gebr32a and BPN14770) to achieve the required cAMP concentration for remyelination, to lie higher than the full inhibitor roflumilast.

Surprisingly, it has been found that the required concentration of a specific PDE4D-inhibitor is lower than of roflumilast. Furthermore, we revealed in vivo a faster functional recovery upon PDE4D inhibition in demyelinated mice compared to vehicle treated mice. In contrast to PDE4D-specific inhibition, the pan-PDE4 inhibitor roflumilast displayed emetic side-effects at the remyelination-inducing dose.

In conclusion, PDE4D-specific inhibition is an innovative and promising approach to boost (re)myelination in demyelinating diseases such as multiple sclerosis without emetic side effects. Therefore, we aim to halt and reverse pMS by boosting remyelination by selectively inhibiting phosphodiesterase type 4D (PDE4D) splice variants as a novel molecular target.

### SUMMARY OF THE INVENTION

The current invention relates to selective PDE4D inhibitor(s) for use in the prevention and/or treatment of demyelinating diseases of the nervous system in a subject. The PDE4D inhibitors of the present invention are typically characterized in that they selectively inhibit the type D isoforms of PDE4. In particular, the selective PDE4D inhibitor of the present invention is further characterized in that it inhibits maximum 45% of the activity of the type A, B and C isoforms of PDE4. In a further embodiment, the selective PDE4 inhibitor of the invention is characterized in that it inhibits at least 50% of the activity of the type D isoforms of PDE4. In an even more preferred embodiment, the selective PDE4 inhibitor of the present invention inhibits at least 60% of the activity of the type D isoforms of PDE4. In still an even more specific embodiment, the selective PDE4 inhibitor of the present invention inhibits maximum 45% of the activity of the type A, B and C isoforms of PDE4 and inhibits at least 50% of the activity of the type D isoforms of PDE4.

In a particular embodiment, the selective PDE4D inhibitor(s) of the present invention are for use in restoring the remyelination process in the treatment of a demyelinating disease of the nervous system in said subject.

In a further embodiment, the demyelinating disease of the nervous system is a demyelinating disease of the central nervous system. Said demyelinating diseases of the central nervous system can be selected from multiple sclerosis (MS), neuromyelitis optic (Devic's disease), inflammatory demyelinating diseases, central nervous system neuropathy, central pontine myelinolysis, myelopathy, leukoencephalopathy, or leukodystrophy. In still a further embodiment, the demyelinating disease of the central nervous system is multiple sclerosis (MS).

In another embodiment, the selective PDE4D inhibitor(s) according to the invention are for use in restoring the remyelination process in the treatment of progressive MS (pMS) of a subject. It is accordingly an objective of the present invention to provide selective PDE4D inhibitor(s) for use in the prevention and/or treatment of progressive MS in a subject; more in particular for use in the prevention and/or treatment of primary progressive multiple sclerosis, secondary progressive multiple sclerosis or relapse remitting multiple sclerosis.

In another aspect, the demyelinating disease of the nervous system is a demyelinating disease of the peripheral nervous system. In a further embodiment, said demyelinating disease of the peripheral nervous system is a demyelinating disease associated with peripheral neuropathy. In an even more preferred embodiment, the demyelinating disease of the peripheral nervous system is selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury.

In all aspects of the present, the subject can be a non-human animal or a human; in a preferred embodiment, the subject is a mammal; in an even more preferred embodiment, the subject is a human.

In a certain embodiment, the selective PDE4D inhibitor(s) that selectively inhibit the type D isoform of PDE4. for use according to the invention, are represented by formula (I),

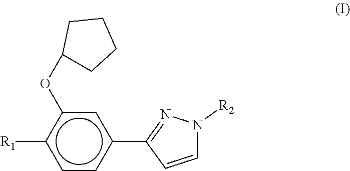

wherein

R₁ and R₂ are independently selected from a group comprising —OH, —NH₂, halo, —C₁₋₈alkyl and C₁₋₈alkoxy-, wherein said —C₁₋₈alkyl and C₁₋₈alkoxy- are optionally substituted with one or more groups selected from —OH, —NH₂, halo, Ar₁ and Het₁.

Ar₁ represents a polyunsaturated, aromatic hydrocarbyl group having a single ring or multiple aromatic rings fused together or linked covalently, typically containing 6 to 10 atoms; wherein at least one ring is aromatic;

Het₁ represents a morpholino ring or a 5 to 12 carbon-atom aromatic ring or ring system containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms; or a salt thereof including a pharmaceutically acceptable salt thereof.

In a further embodiment, the selective PDE4D inhibitors for use according to the invention are represented by Formula (I)

wherein

R₁ is a C₁₋₈ alkoxy- optionally substituted with one or more groups selected from —OH, —NH₂ and halo; more in particular a C₁₋₈alkoxy- optionally substituted with one or more groups selected from halo; more in particular R₁ is a difluoromethoxy;

R₂ is a —C₁₋₈ alkyl optionally substituted with one or more groups selected from —OH and Het₁; Het₁ represents a morpholino ring or a 5 to 6 carbon-atom aromatic ring in which one or more carbon atoms in one or more of these rings can be replaced by oxygen, nitrogen or sulfur atoms; more in particular nitrogen and oxygen; more in particular Het₁ is a morpholino ring; or a salt thereof including a pharmaceutically acceptable salt thereof.

In another embodiment, the selective PDE4D inhibitors for use according to the invention, are represented by Formula (II),

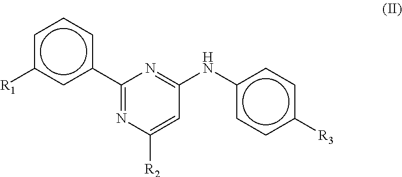

wherein

R₁, R₂ and R₃ are independently selected from a group comprising —OH, —NH₂, halo, —C₁₋₈alkyl, C₁₋₈ alkoxy- and —C₁₋₈ alkylamine wherein said —C₁₋₈alkyl, C₁₋₈alkoxy- and —C₁₋₈ alkylamine are optionally substituted with one or more groups selected from —OH, —NH₂, halo, oxo, Ar₁ and Het₁.

Ar₁ represents a polyunsaturated, aromatic hydrocarbyl group having a single ring or multiple aromatic rings fused together or linked covalently, typically containing 6 to 10 atoms; wherein at least one ring is aromatic;

Het₁ represents a 5 to 12 carbon-atom aromatic ring or ring system containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms;

or a salt thereof including a pharmaceutically acceptable salt thereof.

In a further embodiment, the selective PDE4D inhibitors for use according to the invention are represented by Formula (II),

wherein

R₁ is halo, more in particular R₁ is Cl;

R₂ is a —C₁₋₈ alkyl optionally substituted with one or more halo, more in particular F; more in particular R₂ is —CF₃;

R₃ is a —C₁₋₈ alkylamine optionally substituted with one or more oxo;

or a salt thereof including a pharmaceutically acceptable salt thereof.

In a preferred embodiment, the selective PDE4D inhibitors for use according to the invention are selected from

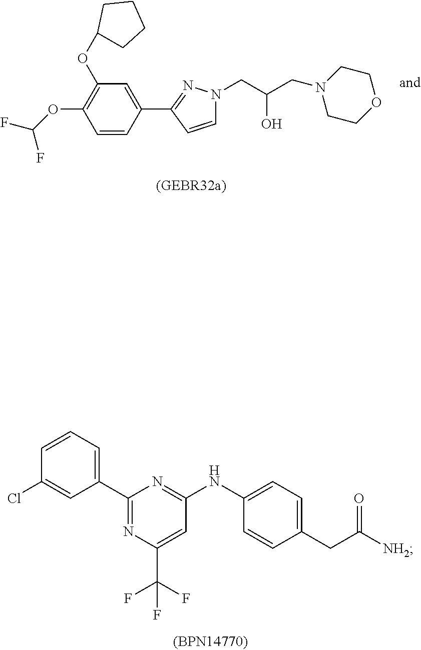 [SMILES: OC(CN1CCOCC1)Cn1ccc(-c2ccc(OC(F)F)c(OC3CCCC3)c2)n1 ; NC(=O)Cc1ccc(Nc2cc(C(F)(F)F)nc(-c3cccc(Cl)c3)n2)cc1]

or a salt thereof including a pharmaceutically acceptable salt thereof.

In a specific embodiment, the selective PDE4D inhibitor(s) are administered at a daily dose rate between 0.01 and 1000 mg, preferably between 0.025 and 750 mg, even more preferably between 0.05 and 500 mg.

Furthermore, the invention provides the use of selective PDE4D inhibitor(s) in in vitro, ex vivo and in vivo remyelination assays.

In another embodiment, the present invention provides a pharmaceutical composition comprising one or more selective PDE4D inhibitor(s) as mentioned above, for use in the diagnosis, prevention and/or treatment of demyelinating diseases; in particular for use in the diagnosis, prevention and/or treatment of demyelinating diseases of the central nervous system or demyelinating diseases of the peripheral nervous system.

In a further embodiment, the demyelinating disease of the nervous system is a demyelinating disease of the central nervous system. Said demyelinating diseases of the central nervous system can be selected from multiple sclerosis (MS), neuromyelitis optic (Devic's disease), inflammatory demyelinating diseases, central nervous system neuropathy, central pontine myelinolysis, myelopathy, leukoencephalopathy, or leukodystrophy. In still a further embodiment, the demyelinating disease of the central nervous system is multiple sclerosis (MS). In still another further embodiment, the demyelinating disease of the central nervous system is progressive multiple sclerosis. Therefore, in an even more preferred embodiment, the present invention provides a pharmaceutical composition comprising one or more selective PDE4D inhibitor(s) for use as a medicament in restoring the remyelination process in the treatment of progressive multiple sclerosis.

In still another embodiment, the demyelinating disease of the nervous system is a demyelinating disease of the peripheral nervous system. In a further embodiment, said demyelinating disease of the peripheral nervous system is a demyelinating disease associated with peripheral neuropathy. In an even more preferred embodiment, the demyelinating disease of the peripheral nervous system is selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy or traumatic nerve injury. Thus, the present invention is also directed to a pharmaceutical composition comprising one or more selective PDE4D inhibitors as described above, for use in the diagnosis, prevention and/or treatment of demyelinating diseases of the peripheral nervous system; preferably demyelinating diseases of the peripheral nervous system associated with peripheral neuropathy; even more preferably selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury.

In another embodiment, the present invention provides a method for preventing and/or treating demyelinating diseases in a subject; in particular demyelinating diseases of the central or peripheral nervous system, said method comprising administering a pharmaceutical composition as described above to said subject. In a further embodiment, the present invention provides a method for preventing and/or treating multiple sclerosis; preferably progressive multiple sclerosis in a subject, comprising administering a pharmaceutical composition as described above to said subject. In an even further embodiment the present invention provides a method for restoring the remyelination process in the treatment of progressive multiple sclerosis in a subject, said method comprising administering a pharmaceutical composition as described above to said subject. I

n another embodiment, the present invention provides a method for preventing and/or treating demyelinating diseases of the peripheral nervous system; preferably demyelinating diseases of the peripheral nervous system associated with peripheral neuropathy. In an even more preferred embodiment, the demyelinating disease of the peripheral nervous system is selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy or traumatic nerve injury.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: qPCR profiles of the PDE4D isoforms (PDE4D1, PDE4D3, PDE4D4, PDE4D5, PDE4D6, PDE4D7, PDE4D8 & PDE4D9) in human oligodendrocytes (OLg) and oligodendrocyte precursor cells (OPC). mRNA of oligodendrocytes (OLg) and oligodendrocyte precursor cells (OPC) was isolated from the human central nervous system as described previously (Cui Q. L. et al, Am J Pathol 2013, 183(2)). Additionally, RNAwas isolated from the area postrema (AP), the center in the brain responsible for emesis. qPCR was performed on paired OLg and OPC and AP using verified primer couples for different PDE4D splice variants (OPC/Oligo: n=3; AP: n=14). The proportional contribution of each splice variant was calculated for each sample (sum isoforms per sample=1).

FIG. 2: qPCR profiles of PDE4D isoforms (PDE4D1, PDE4D3, PDE4D4, PDE4D5, PDE4D6, PDE4D7, PDE4D8 & PDE4D9) in normal appearing white matter (NAWM) and MRI-confirmed chronic inactive multiple sclerosis lesions (MRI). mRNA was isolated from normal appearing white matter (NAWM) and MRI-confirmed chronic inactive multiple sclerosis lesions (MRI). qPCR was performed on NWAM and MRI white matter using verified primer couples for different PDE4D splice variants (n≥5). Because of this within design, the expression profiles of MRI white matter were normalized to the expression within the NAWM. A one sample t-test was performed (MRI expression tested compared to theoretical mean of 1). Data are displayed as mean+/−sem; *P<0.05; **P<0.01.

FIG. 3: Inhibition of PDE4 by roflumilast induces differentiation of primary mouse oligodendrocyte precursor cells. Primary mouse oligodendrocyte precursor cells (OPCs) were isolated from p0 C57bl6 pups using the shake off method at day 0. Primary OPCs (150.000 cells/condition) were cultured and stimulated with vehicle (0.1% DMSO) or the PDE4 inhibitor Roflumilast (5 μM, or 10 μM) in 0.1% DMSO. Treatment was repeated on day 2 and day 4, applying a 40% medium change. Cells were fixated at day 6 and stained for O4, a late OPC marker and MBP, an oligodendrocyte marker.

The MBP expression (A) and the MBP to O4 ratio (B) increased dose-dependently upon PDE4D inhibition. Data (n=4/group) are displayed as mean+/−SEM. For western blot, primary OPCs (500.000 cells/condition) were cultured and stimulated with vehicle (0.1% DMSO) or the PDE4 inhibitor Roflumilast (5 μM) in 0.1% DMSO. Treatment was repeated on day 2 and day 4, applying a 40% medium change. MBP and β-actin protein expression was quantified using image J and the ratio MBP to β-actin is displayed (C). IHC Data were analyzed using a non-parametric Kruskal-Wallis test with Dunn's multiple comparisons test was performed; p<0.05=*; n=4/group. Western blot data were analyzed using a non-parametric Mann-Withney test; p<0.05; n=4/group.

FIG. 4: Inhibition of PDE4D by Gebr32a induces differentiation of primary mouse oligodendrocyte precursor cells. Primary mouse oligodendrocyte precursor cells (OPCs) were isolated from p0 C57bl6 pups using the shake off method at day 0. Primary OPCs (150.000 cells/condition) were cultured and stimulated with vehicle (0.1% DMSO) or the PDE4D inhibitor Gebr32a (0.5 μM, 1 μM or 5 μM) in 0.1% DMSO. Treatment was repeated on day 2 and day 4, applying a 40% medium change. Cells were fixated at day 6 and stained for O4, a late OPC marker and MBP, an oligodendrocyte marker.

The MBP expression (A) and the MBP to O4 ratio (B) increased dose-dependently upon PDE4D inhibition. Data (n=4/group) are displayed as mean+/−SEM. For western blot, primary OPCs (500.000 cells/condition) were cultured and stimulated with vehicle (0.1% DMSO) or the PDE4D inhibitor Gebr32a (5 μM) in 0.1% DMSO. Treatment was repeated on day 2 and day 4, applying a 40% medium change. MBP and β-actin protein expression was quantified using image J and the ratio MBP to β-actin is displayed (C). IHC Data were analyzed using a non-parametric Kruskal-Wallis test with Dunn's multiple comparisons test was performed; p<0.05=*; n=4/group. Western blot data were analyzed using a non-parametric Mann-Withney test; p<0.05; n=4/group.

FIG. 5: Inhibition of PDE4D by BPN14770 induces differentiation of primary mouse oligodendrocyte precursor cells. Primary mouse oligodendrocyte precursor cells (OPCs) were isolated from p0 C57bl6 pups using the shake off method at day 0. Primary OPCs (150.000 cells/condition) were cultured and stimulated with vehicle (PBS) or the PDE4D inhibitor BPN14770 (0.1 μM, 0.3 μM, 1 μM, 3 μM and 10 μM) in PBS. Treatment was repeated on day 2 and day 4, applying a 40% medium change. Cells were fixated at day 6 and stained for O4, a late OPC marker and MBP, an oligodendrocyte marker. The MBP to O4 ratio (A) and the relative MBP expression (B) increased dose-dependently upon PDE4D inhibition. Data (n=2/group) are displayed as mean+/−SEM.

FIG. 6: Inhibition of PDE4 by roflumilast induces (re)myelination in demyelinated brain slices. Mouse brain slices (270 μM) were trimmed from the cerebellum of p10 C57bl6 pups. After 1 week in culture, brain slices were demyelinated using lysolecithin (16 h) and subsequently treated for 14 days with 5 μM roflumilast or vehicle (0.1% DMSO). Treatment was repeated every 2 days with a 60% medium replenishment. Brain slices were fixated using 4% paraformaldehyde and stained for MBP, neurofilament and counterstained with the nuclear DAPI dye. Three slices per animal were quantified for % myelinated axons (n:2/group).

FIG. 7: Inhibition of PDE4D by gebr32a induces (re)myelination in demyelinated brain slices. Mouse brain slices (370 μM) were trimmed from the cerebellum of p10 C57bl6 pups. After 1 week in culture, brain slices were demyelinated using lysolecithin (16 h) and subsequently treated for 14 days with 0.5 μM Gebr32a or vehicle (0.1% DMSO). Treatment was repeated every 2 days with a 60% medium replenishment. Brain slices were fixated using 4% paraformaldehyde and stained for MBP, neurofilament and counterstained with the nuclear DAPI dye. Three slices per animal were quantified for % myelinated axons (n:2/group).

FIG. 8: MBP expression in the corpus callosum upon PDE4 inhibition. A) IHC; B no IHC. Forty-two eleven-weeks-old male C57bl6 mice were either subjected to a cuprizone treatment to induce demyelination (n=32) or left without treatment (control; n=10) for six weeks (day 0-42). From day 40 till day 49, the control mice and part of the cuprizone-treated mice (vehicle; n=10) received a vehicle treatment, receiving a s.c. injection with a 1% DMSO in 0.5% methylcellulose solution twice a day. The remaining cuprizone-treated mice received a roflumilast treatment (roflu 1 mg/kg (n=11) or roflu 3 mg/kg (n=11) in vehicle) twice a day from day 40-49. All animals were sacrificed at day 49. Brains were isolated at anteroposterior coordinates from −0.3 to −1.5 mm was cut in the midsagittal plane and used for slicing and immunohistochemistry (IHC) against MBP. Quantification of the mpb IHC is displayed as mean±standard error of mean.

A one-way ANOVA with Tukey's multiple comparison test showed that the 3 mg/kg roflumilast treated mice had an increased mbp expression, featuring remyelination, compared to the vehicle treated group. The right part of the corpus callosum, anterior of −0.3 was used for qPCR. Besides a reduced mbp mRNA expression in the 1 mg/kg roflumilast treated group compared to the control, no differences were detected at the mRNA level (*p<0.05, **p<0.01, ***p<0.001).

FIG. 9: MBP expression in the hippocampus upon PDE4 inhibition. Forty-two eleven-weeks-old male C57bl6 mice were either subjected to a cuprizone treatment to induce demyelination (n=32) or left without treatment (control; n=10) for six weeks (day 0-42). From day 40 till day 49, the control mice and part of the cuprizone-treated mice (vehicle; n=10) received a vehicle treatment, receiving a s.c. injection with a 1% DMSO in 0.5% methylcellulose solution twice a day. The remaining cuprizone-treated mice received a roflumilast treatment (roflu 1 mg/kg (n=11) or roflu 3 mg/kg (n=11) in vehicle) twice a day from day 40-49. All animals were sacrificed at day 49. Brains were isolated at anteroposterior coordinates from −0.3 to −1.5 mm was cut in the midsagittal plane and used for slicing and immunohistochemistry against MBP. Quantification of the mpb IHC is displayed as mean±standard error of mean. A one-way ANOVA with Tukey's multiple comparison test showed that the 3 mg/kg roflumilast treated mice had an increased mbp expression in the dentate gyrus (DG), but not in the cornu amonis 1 (CA1) or 3 (CA3) (*p<0.05, **p<0.01, ***p<0.001).

FIG. 10: MBP expression in the corpus callosum upon PDE4 inhibition. C57bl6 mice were either subjected to a cuprizone treatment to induce demyelination or left without treatment for six weeks (day 0-42). From day 40 till day 49, the control mice and part of the cuprizone-treated mice received a vehicle treatment, receiving a s.c. injection with a 1% DMSO in 0.5% methylcellulose solution twice a day. The remaining cuprizone-treated mice received a roflumilast treatment (roflu 1 mg/kg or roflu 3 mg/kg in vehicle) twice a day from day 40-49. All animals were sacrificed at day 49. The left part of the corpus callosum at bregma Y-Z was used for TEM and subsequent G ratio measurement. G ratio is the ratio of the inner axonal diameter to the total outer diameter representing myelination of axons (e.g. a higher G ratio represents a demyelination). Both 1 mg/kg and 3 mg/kg roflumilast treatment displayed a re-establishment of the G ratio to control values (A and B). Data are expressed as mean±SEM with. (*p<0.05, **p<0.01, ***p<0.001). Control (n=5), vehicle (n=5), roflu 1 mg/kg (n=7), and roflu 3 mg/kg (n=9).

FIG. 11: Inhibition of PDE4 by roflumilast improves spatial memory upon cuprizone-induced demyelination—a functional measure for remyelination. Forty-two nine-weeks-old male C57bl6 mice were trained for the object location task (OLT) as described previously (Sierksma et al. 2014) (day −14−0). Subsequently, three groups were fed a 0.3% cuprizone diet for 6 weeks to induce demyelination while the control group received a regular chow diet (day 0-42). From day 40 till day 49, control (n=10) and vehicle (n=10) treated animals received a s.c. injection with a 1% DMSO in 0.5% methylcellulose solution or roflumilast (roflu 1 mg/kg (n=11) or roflu 3 mg/kg (n=11) in vehicle) twice a day. All animals were sacrificed at day 49 (A). During the last phase of the cuprizone treatment (demyelination), the OLT was performed at the 3 h inter-trial-interval at day 39.

All cuprizone-treated groups showed an impaired discrimination index (D2 value) while control animals showed intact spatial memory (B).

Next, the OLT was performed during remyelination following cuprizone withdrawal, respectively at 47. The roflumilast treated groups (roflu 3 mg/kg) showed recovery of spatial memory at a level comparable to the performance of the control animals. The vehicle and roflu 1 mg/kg treated cuprizone animals did not show a recovery of the spatial memory (C). Data shown in figure B and C are displayed as mean+/−SEM. A one sample t-test was performed to test for spatial memory (e.g. D2≠0*p<0.05; **p<0.01; ***p<0.001). A one-way ANOVA did not reveal significant differences. All mice not reaching an exploration time of 4 s in either of two trials were excluded from analyses. Extreme values were excluded by means of Dixon's principles of exclusion of extreme values.

FIG. 12: MBP expression in the corpus callosum upon PDE4 inhibition. Thirty-six eleven-weeks-old male C57bl6 mice were either subjected to a cuprizone treatment to induce demyelination (n=27) or left without treatment (control; n=9) for six weeks (day 0-42). From day 40 till day 49, the control mice and part of the cuprizone-treated mice (vehicle; n=9) received a vehicle treatment, receiving a s.c. injection with a 1% DMSO in 0.5% methylcellulose solution twice a day. The remaining cuprizone-treated mice received a gebr32a treatment (gebr32a 0.1 mg/kg (n=9) or gebr32a 0.3 mg/kg (n=9) in vehicle) twice a day from day 40-49. All animals were sacrificed at day 49. Brains were isolated at anteroposterior coordinates from −0.3 to −1.5 mm was cut in the midsagittal plane and used for slicing and immunohistochemistry against MBP. Quantification of the mpb IHC is displayed as mean±standard error of mean. A one-way ANOVA with Tukey's multiple comparison test showed that the 0.3 mg/kg gebr32a treated mice had an increased mbp expression, featuring remyelination, compared to the vehicle treated group (*p<0.05, **p<0.01, ***p<0.001).

FIG. 13. MBP expression in the dendate gyrus upon PDE4D inhibition. Thirty-six eleven-weeks-old male C57bl6 mice were either subjected to a cuprizone treatment to induce demyelination (n=27) or left without treatment (control; n=9) for six weeks (day 0-42). From 20 day 40 till day 49, the control mice and part of the cuprizone-treated mice (vehicle; n=9) received a vehicle treatment, receiving a s.c. injection with a 1% DMSO in 0.5% methylcellulose solution twice a day. The remaining cuprizone-treated mice received a gebr32a treatment (gebr32a 0.1 mg/kg (n=9) or gebr32a 0.3 mg/kg (n=9) in vehicle) twice a day from day 40-49. All animals were sacrificed at day 49. Brains were isolated at anteroposterior coordinates from −0.3 to −1.5 mm was cut in the midsagittal plane and used for slicing and immunohistochemistry on the dendate gyrus against MBP. Quantification of the mpb IHC is displayed as mean±standard error of mean. A one-way ANOVA with Tukey's multiple comparison test showed that the 0.3 mg/kg gebr32a treated mice had an increased mbp expression, featuring remyelination, compared to the vehicle treated group (*p<0.05, **p<0.01).

FIG. 14: Inhibition of PDE4D by Gebr32a improves spatial memory upon cuprizone-induced demyelination—a functional measure for remyelination. Eighty-eight nine-weeks-old male C57bl6 mice were trained for the object location task (OLT; n=22 per group) as described previously (Sierksma et al. 2014) (day −14−0). Subsequently, three groups were fed a 0.3% cuprizone diet for 6 weeks to induce demyelination while the control group received a regular chow diet (day 0-42). From day 40 till day 49, control and vehicle treated animals received a s.c. injection with a 1% DMSO in 0.5% methylcellulose solution or gebr32a (0.1 mg/kg or 0.3 mg/kg in vehicle) twice a day. All animals were sacrificed at day 49 (A).

During the last phase of the cuprizone treatment, the OLT was performed at the 3 h inter-trial-interval at day 31, day 36, and day 39. All cuprizone-treated groups showed an impaired discrimination index (D2 value) while control animals showed intact spatial memory (B).

Next, the OLT was performed during remyelination following cuprizone withdrawal, respectively at day 45 and 47. The Gebr32a treated groups (0.1 mg/kg and 0.3 mg/kg) showed recovery of spatial memory at a level comparable to the performance of the control animals. The vehicle treated cuprizone animals did not show a recovery of the spatial memory (C).

Data shown in figure B and C are displayed as mean+/−SEM and represent an average of the weighted mean of the individual measurements per mouse in respectively de-and remyelination. A one sample t-test was performed to test for spatial memory (e.g. D2=/0; *p<0.05; **p<0.01; ***p<0.001). A one-way ANOVA with a Tukey's multiple comparison test was performed. All mice not reaching an exploration time of 4 s in either of two trials were excluded from analyses. Extreme values were calculated and excluded by means of Dixon's principles of exclusion of extreme values.

FIG. 15: Inhibition of PDE4D by Gebr32a at the repair inducing dose, does not improves disease course in the inflammatory experimental autoimmune encephalomyelitis (EAE) model for MS.

Fifty-six ten-weeks-old female C57bl6 mice were immunized in the flank and neck with 200 μg/mouse MOG35-55 peptide (Hooke) emulsified in Complete Freund's Adjuvant containing Mycobacterium tuberculosis. Immediately after immunization, treatment was started and lasted for 27 days. Treatment consisted of twice a day a s.c. injection with a 1% DMSO in 0.5% methylcellulose solution, roflumilast (0.3 mg/kg or 3 mg/kg in vehicle) or gebr32a (0.3 mg/kg in vehicle). All animals were sacrificed at day 27. Animals were neurologically scored for clinical signs on a daily basis using the following scores: 0=normal behavior, 0.5=distal limp tail, 1=complete limp tail, 2=limp tail and hind leg inhibition, 2.5=limp tail and weakness of hind legs, 3=limp tail and dragging of hind legs, 3.5=limp tail, complete paralysis of hind legs and animal is unable to right itself when placed on its side, 4=limp tail, complete hind leg paralysis and partial front leg paralysis, 4.5=limp tail, complete hind leg paralysis, partial front leg paralysis and no movement around the cage, 5=death.

A: Prophylactic treatment with gebr32a (0.3 mg/kg) has no effect on clinical score of the EAE mice, whereas roflumilast treated animals (0.3 mg/kg and 3 mg/kg) showed a dose-dependent decrease in disease score. A two-way ANOVA with Tukey's multiple comparison test was performed to test for differences

#: Day 16-Day 25: significant difference between control treated animals and animals treated with 3 mg/kg Roflumilast. P<0.05 at day 16, day 18 and day 25. P<0.01 at day 17, day 19, day 23 and day 24. P<0.005 at day 20, day 21 and day 22.

##: Day 19-Day 25: significant difference between animals treated with 0.3 mg/kg Gebr32a and animals treated with 3 mg/kg Roflumilast. P<0.01 at day 19 and day 25. P<0.005 at day 20, day 21, day 22, day 23 and day 24.

###: Day 21-Day 24: significant difference between animals treated with 0.3 mg/kg Roflumilast and animals treated with 3 mg/kg Roflumilast. P<0.05 at day 21, day 22, day 23 and day 24.

FIG. 16: Inhibition of PDE4 by roflumilast and PDE4D by BPN induces differentiation of primary rat schwann cells. Primary rat schwann cells were isolated from p3 Wistar rats. Primary rat schwann cells were cultured onto an aligned network of electrospinned fibers and stimulated with vehicle (0.1% DMSO), the PDE4 inhibitor Roflumilast (5 μM or 10 μM) or the PDE4D inhibitor BPN (1 μM or 5 μM), both dissolved in 0.1% DMSO. Cells were fixated (IHC) or lysated (WB and qPCR) at day 14.

A: Primary rat schwann cells were stained for MBP (red) and MAG (green), both markers for Schwann cells differentiation (100.000 cells). IHC showed that MBP and MAG protein expression was increased dose-dependently upon PDE4 or PDE4D inhibition.

B: MBP and β-actin protein expression of primary rat schwann cells was analyzed using western blot (500.000 cells)

C: qPCR was conducted to evaluate changes in mRNA expression of MBP, PLP, MAG and SOX10. The expression of all these genes was increased upon PDE4 or PDE4D inhibition. Data (n≥8/group are displayed as mean+/−SEM. Data were analyzed using a one-way ANOVA with Dunnett's multiple comparison test (*p<0.05, **p<0.01, ***p<0.001 compared to control conditions).

### DETAILED DESCRIPTION OF THE INVENTION

The present invention is typically characterized in that it provides selective PDE4D inhibitors for use in the treatment of demyelinating diseases. In contrast to pan-PDE4 inhibitors that inhibit all types of isoforms of PDE4D at a high level, the present invention is directed to selective PDE4D inhibitors that selectively inhibit the type D isoform of PDE4. In the context of the present invention, selective inhibition of the type D isoforms of PDE4 is defined as at least 50% inhibition of the activity of the type D isoforms of PDE4 and maximum 45% inhibition of the activity of the other (type A, B and C) isoforms of PDE4. In an even preferred embodiment, selective inhibition of the type D isoform of PDE4 is defined as at least 60% inhibition of the activity of the type D isoforms of PDE4 and maximum 45% inhibition of the activity of the other (type A, B and C) isoforms of PDE4. Thus, in the context of the present invention, selective PDE4D inhibitors are inhibitors that inhibit at least 50% of the activity of type D isoforms of PDE4 and inhibit the activity of the other Type A, B and C isoforms of PDE4 with maximum 45%. Also in the context of the invention, non-selective PDE4 inhibitors (or pan-PDE4 inhibitors) are inhibitors of PDE4 that inhibit all isoforms of PDE4 to a large degree.

The inventors surprisingly found that selective PDE4D inhibitors, such as for example Gebr32a and BPN 14770, stimulated the differentiation of oligodendrocytes in vitro and improved (re)myelination in ex vivo demyelinated cerebellar brain slices. Remarkably, and in contrast to the pan-PDE4 inhibitors, such as roflumilast, only low doses of the selective PDE4D inhibitors are sufficient to achieve their effect, and hence, emetic side-effects, that are often observed after treatment with pan-PDE4 inhibitors, are absent. This might be in contrast to what would be expected by a skilled person, since the selective PDE4D inhibitors of the present invention only inhibit the type D isoform of PDE4. Furthermore, the inventors revealed that selective PDE4D inhibition leads to a faster functional recovery in demyelinated mice, without inducing emetic side-effects. In contrast, treatment with the pan-PDE4 inhibitor roflumilast displayed emetic side-effects at the remyelination-inducing dose.

In a further aspect, the inventors found that the selective PDE4D inhibitor Gebr32a did not improve the disease score in the inflammatory experimental autoimmune encephalomyelitis (EAE) model, in contrast to the pan-PDE4 inhibitor roflumilast. This indicates that the selective PDE4D inhibitors of the present invention do not have any anti-inflammatory effects in demyelinating diseases, but that they are able to restore the remyelination directly in demyelinating diseases. This is in sharp contrast to the pan-PDE4 inhibitors such as roflumilast. Furthermore, the inventors also found that the selective PDE4D inhibitors of the present invention are able to induce peripheral myelination by stimulating differentiation of Schwann cells.

The present invention is therefore directed to selective PDE4D inhibitor(s) that selectively inhibit the type D isoform of PDE4 for use in the prevention and/or treatment of demyelinating diseases of the central or peripheral nervous system. In a further embodiment, the selective PD4D inhibitor is for use in restoring the remyelination process in the treatment of a demyelinating disease of the central or peripheral nervous system.

In a further aspect, the invention is directed to said selective PDE4D inhibitors for use in the prevention and/or treatment of multiple sclerosis, wherein the selective PDE4D inhibitor(s) restore the remyelination process in the treatment of MS of said subject.

In another embodiment, the selective PDE4D inhibitor(s) for use according to the invention, restore the remyelination process in the treatment of progressive MS (pMS) of said subject; it is accordingly an objective of the present invention to provide selective PDE4D inhibitor(s) for use in the prevention and/or treatment of progressive MS in a subject, more in particular for use in the prevention and/or treatment of primary progressive multiple sclerosis, secondary progressive multiple sclerosis or relapse remitting multiple sclerosis.

As used herein, the term “demyelinating disease”, is a disease condition in which the myelin sheath which surrounds neurons in nervous tissue is lost or damaged, leading to axonal degeneration and impaired signal transduction in the affected nerves. A demyelinating disease of the central nervous system is a disease in which the myelin sheaths of neurons in the central nervous system are lost or damaged. Examples of demyelinating diseases of the central nervous systems are multiple sclerosis, neuromyelitis optic (Devic's disease), inflammatory demyelinating diseases, central nervous system neuropathy, central pontine myelinolysis, myelopathy, leukoencephalopathy, or leukodystrophy.

A demyelinating disease of the peripheral nervous system is a disease condition in which the myelin sheaths of neurons in the peripheral nervous system are lost or damaged. Examples of demyelinating diseases of the peripheral nervous system are Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury.

As used herein, the term “multiple sclerosis” or “MS” entails an autoimmune-mediated process in which an abnormal response of the body's immune system is directed against the central nervous system (CNS), which is made up of the brain, spinal cord and optic nerves. The immune reaction results in death of oligodendrocytes, demyelination, and eventually loss of axons, featured by a physical and cognitive disability.

As used herein, the term “progressive multiple sclerosis” or “pMS” is featured by an accumulation of chronic demyelinated lesions and is subdivided in Primary progressive MS (PPMS), Secondary progressive MS (SPMS) and relapse remitting MS (RRMS).

Primary progressive MS (PPMS) is characterized by worsening neurologic function (accumulation of disability) from the onset of symptoms, without early relapses or remissions. PPMS can be further characterized at different points in time as either active (with an occasional relapse and/or evidence of new MRI activity) or not active, as well as with progression (evidence of disease worsening on an objective measure of change over time, with or without relapse or new MRI activity) or without progression.

Secondary progressive MS (SPMS) follows an initial relapsing-remitting course. Most people who are diagnosed with a relapse remitting MS (RRMS) will eventually transition to a secondary progressive course in which there is a progressive worsening of neurologic function (accumulation of disability) over time. SPMS can be further characterized at different points in time as either active (with relapses and/or evidence of new MRI activity) or not active, as well as with progression (evidence of disease worsening on an objective measure of change over time, with or without relapses) or without progression.

The subject may be a non-human animal or a human.

In a particular embodiment, the selective PDE4D inhibitors for use according to the invention, are represented by formula (I),

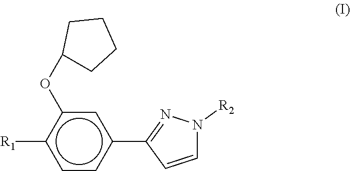

wherein

R₁ and R₂ are independently selected from a group comprising —OH, —NH₂, halo, —C₁₋₈ alkyl and C₁₋₈ alkoxy-, wherein said —C₁₋₈ alkyl and C₁₋₈ alkoxy- are optionally substituted with one or more groups selected from —OH, —NH₂, halo, Ar₁ and Het₁.

Ar₁ (aryl 1) represents a polyunsaturated, aromatic hydrocarbyl group having a single ring or multiple aromatic rings fused together or linked covalently, typically containing 6 to 10 atoms; wherein at least one ring is aromatic;

Het₁ (heteroaryl 1) represents a morpholino ring or a 5 to 12 carbon-atom aromatic ring or ring system containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms; more in particular nitrogen and oxygen; more in particular Het₁ is a morpholino ring; or a salt thereof including a pharmaceutically acceptable salt thereof.

Particular embodiments of the selective PDE4D inhibitors of formula (I), are those wherein one or more of the following restrictions apply;
R₁ and R₂ are independently selected from a group comprising —OH, —NH₂, halo, —C₁₋₈alkyl and C₁₋₈alkoxy-, wherein said —C₁₋₈alkyl and C₁₋₈alkoxy- are optionally substituted with one or more groups selected from —OH, —NH₂, halo, Ar₁ and Het₁;
R₁ is a C₁₋₈ alkoxy- and R₂ is selected from a group comprising —OH, —NH₂, halo, —C₁₋₈alkyl and C₁₋₈ alkoxy-, wherein said —C₁₋₈ alkyl and C₁₋₈ alkoxy- are optionally substituted with one or more groups selected from —OH, —NH₂, halo, Ar₁ and Het₁;
R₂ is a —C₁₋₈ alkyl and R₁ is selected from a group comprising —OH, —NH₂, halo, —C₁₋₈ alkyl and C₁₋₈ alkoxy-, wherein said —C₁₋₈ alkyl and C₁₋₈ alkoxy- are optionally substituted with one or more groups selected from —OH, —NH₂, halo, Ar₁ and Het₁;
R₁ is a C₁₋₈ alkoxy- optionally substituted with one or more halo groups; and R₂ is selected from a group comprising —OH, —NH₂, halo, —C₁₋₈ alkyl and C₁₋₈ alkoxy-, wherein said —C₁₋₈ alkyl and C₁₋₈ alkoxy- are optionally substituted with one or more groups selected from —OH, —NH₂, halo, Ar₁ and Het₁;
R₂ is a —C₁₋₈ alkyl optionally substituted with one or more groups selected from —OH and Het₁; and R₁ is selected from a group comprising —OH, —NH₂, halo, —C₁₋₈ alkyl and C₁₋₈alkoxy-, wherein said —C₁₋₈ alkyl and C₁₋₈ alkoxy- are optionally substituted with one or more groups selected from —OH, —NH₂, halo, Ar₁ and Het₁;
R₁ is a difluoromethoxy; and R₂ is selected from a group comprising —OH, —NH₂, halo, —C₁₋₈ alkyl and C₁₋₈ alkoxy-, wherein said —C₁₋₈ alkyl and C₁₋₈ alkoxy- are optionally substituted with one or more groups selected from —OH, —NH₂, halo, Ar₁ and Heh;
R₁ is a difluoromethoxy; and R₂ is a —C₁₋₈ alkyl substituted with one or more groups selected from —OH and morfoline;

In another embodiment, the selective PDE4D inhibitors for use according to the invention, are represented by formula (II),

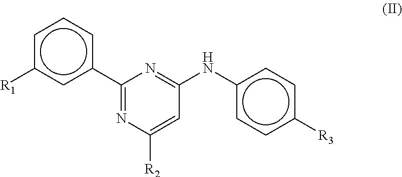

wherein

R₁, R₂ and R₃ are independently selected from a group comprising —OH, —NH₂, halo, —C₁₋₈ alkyl, C₁₋₈ alkoxy- and —C₁₋₈ alkylamine wherein said —C₁₋₈alkyl, C₁₋₈ alkoxy- and —C₁₋₈ alkylamine are optionally substituted with one or more groups selected from —OH, —NH₂, halo, oxo, Ar₁ and Het₁.

Ar₁ represents a polyunsaturated, aromatic hydrocarbyl group having a single ring or multiple aromatic rings fused together or linked covalently, typically containing 6 to 10 atoms; wherein at least one ring is aromatic;

Het₁ represents a 5 to 12 carbon-atom aromatic ring or ring system containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms;

or a salt thereof including a pharmaceutically acceptable salt thereof.

Particular embodiments of the selective PDE4D inhibitors of formula (II), are those wherein one or more of the following restrictions apply;
R₁, R₂ and R₃ are independently selected from a group comprising-OH, —NH₂, halo, —C₁₋₈alkyl, C₁₋₈ alkoxy- and —C₁₋₈ alkylamine wherein said —C₁₋₈ alkyl, C₁₋₈ alkoxy- and —C₁₋₈alkylamine are optionally substituted with one or more groups selected from —OH, —NH₂, halo, oxo, Ar₁ and Het₁.
R₁ is halo; R₂ and R₃ are independently selected from a group comprising —OH, —NH₂, halo, —C₁₋₈ alkyl, C₁₋₈ alkoxy- and —C₁₋₈ alkylamine wherein said —C₁₋₈ alkyl, C₁₋₈ alkoxy- and —C₁₋₈ alkylamine are optionally substituted with one or more groups selected from —OH, —NH₂, halo, oxo, Ar₁ and Het₁.
R₂ is a —C₁₋₈ alkyl, R₁ and R₃ are independently selected from a group comprising —OH, —NH₂, halo, —C₁₋₈ alkyl, C₁₋₈ alkoxy- and —C₁₋₈alkylamine wherein said —C₁₋₈ alkyl, C₁₋₈ alkoxy- and —C₁₋₈ alkylamine are optionally substituted with one or more groups selected from —OH, —NH₂, halo, oxo, Ar₁ and Het₁.
R₃ is C₁₋₈ alkylamine, R₁ and R₂ are independently selected from a group comprising —OH, —NH₂, halo, —C₁₋₈ alkyl, C₁₋₈alkoxy- and —C₁₋₈ alkylamine wherein said —C₁₋₈ alkyl, C₁₋₈alkoxy- and —C₁₋₈ alkylamine are optionally substituted with one or more groups selected from —OH, —NH₂, halo, oxo, Ar₁ and Het₁.
R₂ is a —C₁₋₈ alkyl optionally substituted with one or more halo, R₁ and R₃ are independently selected from a group comprising —OH, —NH₂, halo, —C₁₋₈ alkyl, C₁₋₈ alkoxy- and —C₁₋₈ alkylamine wherein said —C₁₋₈ alkyl, C₁₋₈ alkoxy- and —C₁₋₈ alkylamine are optionally substituted with one or more groups selected from —OH, —NH₂, halo, oxo, An and Het₁.
R₃ is C₁₋₈ alkylamine optionally substituted with one or more oxo, R₁ and R₂ are independently selected from a group comprising —OH, —NH₂, halo, —C₁₋₈ alkyl, C₁₋₈ alkoxy- and —C₁₋₈ alkylamine wherein said —C₁₋₈ alkyl, C₁₋₈ alkoxy- and —C₁₋₈ alkylamine are optionally substituted with one or more groups selected from —OH, —NH₂, halo, oxo, An and Het₁.
R₁ is Cl; R₂ is a —C₁₋₈ alkyl optionally substituted with one or more halo, more in particular F; more in particular R₂ is —CF₃; R₃ is a —C₁₋₈ alkylamine optionally substituted with one or more oxo;

In a preferred embodiment, the selective PDE4D inhibitors for use according to the invention are selected from

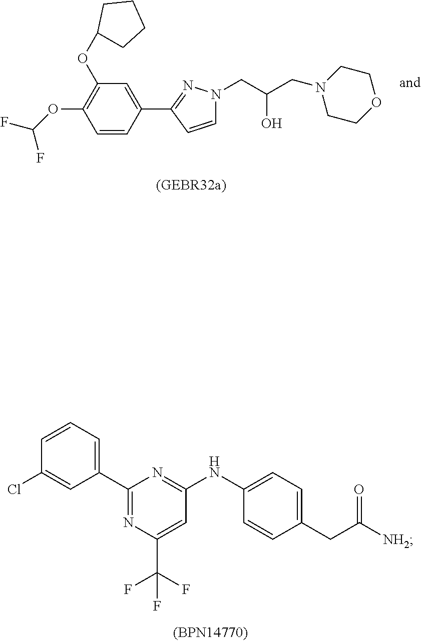 [SMILES: OC(CN1CCOCC1)Cn1ccc(-c2ccc(OC(F)F)c(OC3CCCC3)c2)n1 ; NC(=O)Cc1ccc(Nc2cc(C(F)(F)F)nc(-c3cccc(Cl)c3)n2)cc1]

or a salt thereof including a pharmaceutically acceptable salt thereof.

Compounds of formula I, in particular Gebr32a may be prepared as described in the experimental procedures WO2015121212, in particular scheme 8 of WO2015121212. Compounds of formula II, in particular BPN14770 may be prepared as described in the examples of WO2014066659, in particular example 220 of WO2014066659.

The term “alkyl” by itself or as part of another substituent refers to a fully saturated hydrocarbon of Formula CₓH₂ₓ₊₁ wherein x is a number greater than or equal to 1. Generally, alkyl groups of this invention comprise from 1 to 20 carbon atoms. Alkyl groups may be linear or branched and may be substituted as indicated herein. When a subscript is used herein following a carbon atom, the subscript refers to the number of carbon atoms that the named group may contain. Thus, for example, C₁₋₄alkyl means an alkyl of one to four carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers, heptyl and its isomers, octyl and its isomers, nonyl and its isomers; decyl and its isomers. C₁-C₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopentyl, 2-, 3-, or 4-methylcyclopentyl, cyclopentylmethylene, and cyclohexyl.

The term “optionally substituted” refers to a certain group optionally substituted with one or more substituents (for example 1 to 4 substituents, for example 1, 2, 3, or 4 substituents or 1 to 2 substituents) at any available point of attachment. Non-limiting examples of such substituents include halo, hydroxyl, carbonyl, nitro, amino, oxime, imino, azido, hydrazino, cyano, aryl, heteroaryl, cycloalkyl, acyl, alkylamino, alkoxy, thiol, alkylthio, carboxylic acid, acylamino, alkyl esters, carbamate, thioamido, urea, sullfonamido and the like.

The term “alkoxy” or “alkyloxy” as used herein refers to a radical having the Formula —OR^{b} wherein R^{b} is alkyl. Preferably, alkoxy is C₁-C₁₀ alkoxy, C₁-C₆ alkoxy, or C₁-C₄ alkoxy. Non-limiting examples of suitable alkoxy include methoxy, ethoxy, propoxy, isopropoxy, butoxy, isobutoxy, sec-butoxy, tert-butoxy, pentyloxy and hexyloxy. Where the oxygen atom in an alkoxy group is substituted with sulfur, the resultant radical is referred to as thioalkoxy. “Haloalkoxy” is an alkoxy group wherein one or more hydrogen atoms in the alkyl group are substituted with halogen. Non-limiting examples of suitable haloalkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy; trichloromethoxy, 2-bromoethoxy, pentafluoroethyl, 3,3,3-trichloropropoxy, 4,4,4-trichlorobutoxy.

The term “alkylamine” as used herein refers to an alkyl as defined above comprising a —NH₂.

The term “aryl” as used herein refers to a polyunsaturated, aromatic hydrocarbyl group having a single ring (i.e. phenyl) or multiple aromatic rings fused together (e.g. naphthalene or anthracene) or linked covalently, typically containing 6 to 10 atoms; wherein at least one ring is aromatic. The aromatic ring may optionally include one to three additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7-, or 8-azulenyl, 1- or 2-naphthyl, 1-, 2-, or 3-indenyl, 1-, 2-, or 9-anthryl, 1-2-, 3-, 4-, or 5-acenaphtylenyl, 3-, 4-, or 5-acenaphtenyl, 1-, 2-, 3-, 4-, or 10-phenanthryl, 1- or 2-pentalenyl, 1, 2-, 3-, or 4-fluorenyl, 4- or 5-indanyl, 5-, 6-, 7-, or 8-tetrahydronaphthyl, 1,2,3,4-tetrahydronaphthyl, 1,4-dihydronaphthyl, dibenzo[a,d]cylcoheptenyl, and 1-, 2-, 3-, 4-, or 5-pyrenyl.

The term “heteroaryl” as used herein by itself or as part of another group refers but is not limited to 5 to 12 carbon-atom aromatic rings or ring systems containing 1 to 3 rings which are fused together or linked covalently, typically containing 5 to 8 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, benzopyranyl, 1(4H)-benzopyranyl, 1(2H)-benzopyranyl, 3,4-dihydro-1(2H)-benzopyranyl, 3,4-dihydro-1(2H)-benzopyranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl, 7-azaindolyl, 6-azaindolyl, 5-azaindolyl, 4-azaindolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl; oxazol-4-yl and oxazol-5-yl. The term “isoxazolyl” (also called 1,2-oxazolyl), as used herein includes isoxazol-3-yl, isoxazol-4-yl, and isoxazol-5-yl. The term “thiazolyl” (also called 1,3-thiazolyl), as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2, 1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl,-indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein includes isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quiriazolin-4-yl, quinazolin-5-yl, quinazolin-6-yl, quinazolin-7-yl and quinazolin-8-yl. The term “quinoxalinyl”. as used herein includes quinoxalin-2-yl, quinoxalin-5-yl, and quinoxalin-6-yl. The term “7-azaindolyl” as used herein refers to 1H-Pyrrolo[2,3-b]pyridinyl and includes 7-azaindol-1-yl, 7-azaindol-2-yl, 7-azaindol-3-yl, 7-azaindol-4-yl, 7-azaindol-5-yl, 7-azaindol-6-yl. The term “6-azaindolyl” as used herein refers to 1H-Pyrrolo[2,3-c]pyridinyl and includes 6-azaindol-1-yl, 6-azaindol-2-yl, 6-azaindol-3-yl, 6-azaindol-4-yl, 6-azaindol-5-yl, 6-azaindol-7-yl. The term “5-azaindolyl” as used herein refers to 1H-Pyrrolo[3,2-c]pyridinyl and includes 5-azaindol-1-yl, 5-azaindol-2-yl, 5-azaindol-3-yl, 5-azaindol-4-yl, 5-azaindol-6-yl, 5-azaindol-7-yl. The term “4-azaindolyl” as used herein refers to 1H-Pyrrolo[3,2-b]pyridinyl and includes 4-azaindol-1-yl, 4-azaindol-2-yl, 4-azaindol-3-yl, 4-azaindol-5-yl, 4-azaindol-6-yl, 4-azaindol-7-yl.

For example, non-limiting examples of heteroaryl can be 2- or 3-furyl, 2- or 3-thienyl, 1-, 2- or 3-pyrrolyl, 1-, 2-, 4- or 5-imidazolyl, 1-, 3-, 4- or 5-pyrazolyl, 3-, 4- or 5-isoxazolyl, 2-, 4- or 5-oxazolyl, 3-, 4- or 5-isothiazolyl, 2-, 4- or 5-thiazolyl, 1,2,3-triazol-1-, -4- or -5-yl, 1,2,4-triazol-1-, -3-, -4- or -5-yl, 1H-tetrazol-1-, or -5-yl, 2H-tetrazol-2-, or -5-yl, 1,2,3-oxadiazol-4- or -5-yl, 1,2,4-oxadiazol-3- or -5-yl, 1,2,5-oxadiazolyl, 1,3,4-oxadiazolyl, 1,2,3-thiadiazol-4- or -5-yl, 1,2,4-thiadiazol-3- or -5-yl, 1,2,5-thiadiazol-3- or -4-yl, 1,3,4-thiadiazolyl, 1- or 5-tetrazolyl, 2-, 3- or 4-pyridyl, 3- or 4-pyridazinyl, 2-, 4-, 5- or 6-pyrimidyl, 2-, 3-, 4-, 5-6-2H-thiopyranyl, 2-, 3- or 4-4H-thiopyranyl, 4-azaindol-1-, 2-, 3-, 5-, or 7-yl, 5-azaindol-1-, or 2-, 3-, 4-, 6-, or 7-yl, 6-azaindol-1, 2-, 3-, 4-, 5-, or 7-yl, 7-azaindol-1-, 2-, 3-, 4, 5-, or 6-yl, 2-, 3-, 4-, 5-, 6- or 7-benzofuryl, 1-, 3-, 4- or 5-isobenzofuryl, 2-, 3-, 4-, 5-, 6- or 7-benzothienyl, 1-, 3-, 4- or 5-isobenzothienyl, 1-, 2-, 3-, 4-, 5-, 6- or 7-indolyl, 2- or 3-pyrazinyl, 1,4-oxazin-2- or -3-yl, 1,4-dioxin-2- or -3-yl, 1,4-thiazin-2- or -3-yl, 1,2,3-triazinyl, 1,2,4-triazinyl, 1,3,5-triazin-2-, -4- or -6-yl, thieno[2,3-b]furan-2-, -3-, -4-, or -5-yl, benzimidazol-1-yl, -2-yl, -4-yl, -5-yl, -6-yl, or -7-yl, 1-, 3-, 4-, 5-, 6- or 7-benzopyrazolyl, 3-, 4-, 5-, 6- or 7-benzisoxazolyl, 2-, 4-, 5-, 6- or 7-benzoxazolyl, 3-, 4-, 5-, 6- or 7-benzisothiazolyl, 1,3-benzothiazol-2-yl, -4-yl, -5-yl, -6-yl or -7-yl, 1,3-benzodioxol-4-yl, -5-yl, -6-yl, or -7-yl, benzotriazol-1-yl, -4-yl, -5-yl, -6-yl or -7-yl1-, 2-thianthrenyl, 3-, 4- or 5-isobenzofuranyl, 1-, 2-, 3-, 4- or 9-xanthenyl, 1-, 2-, 3- or 4-phenoxathiinyl, 2-, 3-pyrazinyl, 1-, 2-, 3-, 4-, 5-, 6-, 7- or 8-indolizinyl, 2-, 3-, 4- or 5-isoindolyl, 1H-indazol-1-yl, 3-yl, -4-yl, -5-yl, -6-yl, or -7-yl, 2H-indazol-2-yl, 3-yl, -4-yl, -5-yl, -6-yl, or -7-yl, imidazo[2,1-b][1,3]thiazoi-2-yl, imidazo[2,1-b][1,3]thiazol-3-yl, imidazo[2, 1-b][1,3]thiazol-5-yl or imidazo[2,1-b][1,3]thiazol-6-yl, imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl or imidazo[1,2-a]pyridin-7-yl, tetrazolo[1,5-a]pyridine-5-yl, tetrazolo[1,5-a]pyridine-6-yl, tetrazolo[1,5-a]pyridine-7-yl, or tetrazolo[1,5-a]pyridine-8-yl, 2-, 6-, 7- or 8-purinyl, 4-, 5- or 6-phthalazinyl, 2-, 3- or 4-naphthyridinyl, 2-, 5- or 6-quinoxalinyl, 2-, 4-, 5-, 6-, 7- or 8-quinazolinyl, 1-, 2-, 3- or 4-quinolizinyl, 2-, 3-, 4-, 5-, 6-, 7-, or 8-quinolinyl(quinolyl), 2-, 4-, 5-, 6-, 7- or 8-quinazolyl, 1-, 3-, 4-, 5-, 6-, 7- or 8-isoquinolinyl(isoquinolyl), 3-, 4-, 5-, 6-, 7- or 8-cinnolinyl,2-, 4-, 6- or 7-pteridinyl, 1-, 2-, 3-, 4- or 9-carbazolyl, 1-, 2-, 3-, 4-, 5-, 6-, 7-, 8- or 9-carbolinyl, 1-, 2-, 3-, 4-, 5-, 6-, 7-, 8-, 9- or 10-phenanthridinyl, 1-, 2-, 3- or 4-acridinyl, 1-, 2-, 3-, 4-, 5-, 6-, 7-, 8- or 9-perimidinyl, 2-, 3-, 4-, 5-, 6-, 7-, 8-, 9- or 10-(1,7)phenanthrolinyl, 1- or 2-phenazinyl, 1-, 2-, 3-, 4-, or 10-phenothiazinyl, 3- or 4-furazanyl, 1-, 2-, 3-, 4-, or 10-phenoxazinyl, or additionally substituted derivatives thereof.

The term “oxo” as used herein refers to the group ═O.

The term “halo” or “halogen” as a group or part of a group is generic for fluoro, chloro, bromo, or iodo.

In a specific embodiment, the selective PDE4D inhibitor(s) are administered at a daily dose rate between 0.01 and 1000 mg, preferably between 0.025 and 750 mg, even more preferably between 0.05 and 500 mg.

Furthermore, the invention provides the use of selective PDE4D inhibitors in in vitro, ex vivo and in vivo remyelination assays.

Said in vitro, ex vivo and in vivo remyelination assays may for example be characterized by OPC differentiation assays (in vitro), brain slices (ex vivo) and cuprizone modelling with a molecular and functional readout (in vivo).

In another embodiment, the present invention provides a pharmaceutical composition comprising selective PDE4D inhibitor(s) as mentioned above, for use as a medicament in the diagnosis, prevention or treatment of demyelinating diseases of the nervous system; in particular for use in the diagnosis, prevention and/or treatment of demyelinating diseases of the central nervous system or demyelinating diseases of the peripheral nervous system. Demyelinating diseases of the central nervous system can be multiple sclerosis, neuromyelitis optic (Devic's disease), inflammatory demyelinating diseases, central nervous system neuropathy, central pontine myelinolysis, myelopathy, leukoencephalopathy, or leukodystrophy. In a further embodiment, the demyelinating disease of the central nervous system is multiple sclerosis. In still another further embodiment, the demyelinating disease of the central nervous system is progressive multiple sclerosis. Therefore, in an even more preferred embodiment, the present invention provides a pharmaceutical composition comprising one or more selective PDE4D inhibitor(s) for use as a medicament in restoring the remyelination process in the treatment of progressive multiple sclerosis.

In still another embodiment, the demyelinating disease of the peripheral nerve system is selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury. Thus, the present invention is also directed to a pharmaceutical composition comprising one or more selective PDE4D inhibitors as described above, for use in the diagnosis, prevention and/or treatment of demyelinating diseases of the peripheral nervous system; preferably demyelinating diseases of the peripheral nervous system selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury.

In another embodiment, the present invention provides a method for preventing and/or treating demyelinating diseases of the nervous system in a subject; in particular demyelinating diseases of the central or peripheral nervous system, said method comprising administering a pharmaceutical composition as described above to said subject. In a further embodiment, the present invention provides a method for preventing and/or treating multiple sclerosis; preferably progressive multiple sclerosis in a subject, comprising administering a pharmaceutical composition as described above to said subject. In an even further embodiment the present invention provides a method for restoring the remyelination process in the treatment of a demyelinating disease of the nervous system; preferably a demyelinating disease of the central nervous system; more preferably multiple sclerosis; even more preferably progressive multiple sclerosis in a subject, said method comprising administering a pharmaceutical composition as described above to said subject. In another embodiment, the present invention provides a method for preventing and/or treating demyelinating diseases of the peripheral nervous system; preferably demyelinating diseases of the peripheral nervous system selected from Guillain-Barré syndrome, chronic inflammatory demyelinating polyneuropathy, anti-MAG peripheral neuropathy, Charcot-Marie tooth disease, hereditary neuropathy with liability to pressure palsy; copper deficiency-associated conditions such as peripheral neuropathy, myelopathy, optic neuropathy; progressive inflammatory neuropathy, diabetic neuropathy or traumatic nerve injury, said method comprising administering a pharmaceutical composition as described above to a subject.

Selective PDE4D inhibitory compounds for use in the aforementioned indications, can be identified using a PDE4 inhibition assay known in the art. PDE4D inhibition assays can be performed for example using recombinant human PDE enzymes expressed in a baculoviral system. The preliminary screening assays can be performed by the IMAP technology (Molecular Devices), which is based on the high affinity binding of phosphate by immobilized metal coordination complexes on nanoparticles. The binding reagent complexes with phosphate groups on nucleotide monophosphate generated from cyclic nucleotides (cAMP) through phosphodiesterases. With fluorescence polarization detection, binding causes a change in the rate of the molecular motion of the phosphate bearing molecule and results in an increase in the fluorescence polarization value observed for the fluorescent label attached to the substrate. Rolipram can be used as reference compound. All compounds can be solved in DMSO at 10⁻² M concentration and then diluted with water to the final suitable concentrations. All synthesized compounds can be tested preliminary on PDE4D3 at 10⁻⁵ M concentration, in duplicate. Results showing an inhibition of the control higher than 50% are considered to represent significant effects of the test compounds. IC₅₀ values of less than 10 μM are considered to be potent PDE4 inhibitors (Li et al, 2013).

Compounds showing inhibition control higher than 50% on PDE4D can be further tested on the same isoform enzyme at five concentrations in the interval 10⁻⁸-10⁻⁴ M. IC50 values for rolipram and tested compounds can be determined by nonlinear regression analysis of its inhibition curve, using Hill equation curve fitting (Graph Pad Prism software). IC50 values are reported at μM concentration.

Said inhibition may be effected in vitro, ex vivo and/or in vivo, and when effected in vivo, is preferably effected in a selective manner, as defined above.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. Nos. 6,372,778, 6,369,086, 6,369,087 and 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl-bromides and others. Other pharmaceutically acceptable salts include the sulfate salt ethanolate and sulfate salts.

Generally, for pharmaceutical use, the compounds of the inventions may be formulated as a pharmaceutical preparation or pharmaceutical composition comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), etc. Such suitable administration forms —which may be solid, semi-solid or liquid, depending on the manner of administration—as well as methods and carriers, diluents and excipients for use in the preparation thereof, will be clear to the skilled person; reference is again made to for instance U.S. Pat. Nos. 6,372,778, 6,369,086, 6,369,087 and 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives. An interesting way of formulating the compounds in combination with a cyclodextrin or a derivative thereof has been described in EP-A-721,331. In particular, the present invention encompasses a pharmaceutical composition comprising an effective amount of a compound according to the invention with a pharmaceutically acceptable cyclodextrin.

In addition, co-solvents such as alcohols may improve the solubility and/or the stability of the compounds. In the preparation of aqueous compositions, addition of salts of the compounds of the invention can be more suitable due to their increased water solubility.

Particular reference is made to the compositions, formulations (and carriers, excipients, diluents, etc. for use therein), routes of administration etc., such as those described in WO2015121212. More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 0.01 and 1000 mg, usually between 0.05 and 500 mg, of at least one compound of the invention, e.g. about 0.05, 1, 2.5, 5, 10, 20, 50, 100, 150, 200, 250 or 500 mg per unit dosage.

The compounds can be administered by a variety of routes including the oral, rectal, ocular, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, upon suitable administration, is sufficient to achieve the desired therapeutic or prophylactic effect in the individual to which it is administered.

Usually, depending on the condition to be prevented or treated and the route of administration, such an effective amount will usually be between 0.01 to 1000 mg per day, more often between 0.05 and 500 mg, such as for example about 0.05, 1, 2.5, 5, 10, 20, 50, 100, 150, 200, 250 mg or 500 mg, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

For subcutaneous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

The compositions are of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal. Thus, in a further aspect, the invention relates to a composition for veterinary use that contains at least one compound of the invention and at least one suitable carrier (i.e. a carrier suitable for veterinary use). The invention also relates to the use of a compound of the invention in the preparation of such a composition.

The invention will now be illustrated by means of the following synthetic and biological examples, which do not limit the scope of the invention in any way.

### EXAMPLES

In the presentation of the experimental results and figures, the specific PDE4D inhibitor Gebr32a is used herein as “Gebr32a” or as “Gebr”. Both abbreviations are used and refer to the same inhibitor Gebr32a.

The specific PDE4D inhibitor BPN14770 is used herein as ‘BPN14770” or as “BPN”. Both abbreviations are used and refer to the same inhibitor BPN 14770.

The pan-PDE4 inhibitor roflumilast is sometimes abbreviated as ‘Roflu’. Both the terms “roflumilast” and “roflu” are used and refer to the same inhibitor roflumilast.

Materials and Methods

Animals

51 nine-weeks old (Roflumilast study) and 112 eight-weeks old (Gebr32a study) male C57BI/6J OlaHsd mice (Envigo, Venray (NL)), were kept in a reversed 12 h light/dark cycle. Mice were housed individually in standard open cages in an air-conditioned room with a fixed temperature of 21-22° C. and a humidity of 22-60%. A radio provided continuous background noise. Mice had free access to water and food and were monitored 5 times per week for their weight evolution. All procedures and experiments were approved by the local ethical committee of the University of Hasselt and met the EU guidelines acquired for working with experimental animals.

Cuprizone Inducing Demyelination and Treatment

Roflumilast Study

At the start of the experiment, all animals were phenotyped for baseline cognitive performance in the object location task. Afterwards, 4 groups were defined (n1=13, n2=16, n3=11, n4=11) and group 2, 3 and 4 were subjected to a 0.3% w/w cuprizone diet (Bis(cyclohexanone)-oxaldihydrazone) (Sigma-Aldrich, United States) for 42 days. All groups were phenotyped for cognitive performance at the end of the demyelination phase, preceding treatment. For intermediate post mortem analysis, 3 animals of group 1 (no cuprizone) and 5 animals of group 2 (cuprizone) were sacrificed by an intraperitoneal injection of sodium dolethal (200 mg/kg) followed by transcardial perfusion (10 U/ml heparin in 1×PBS). Subcutaneous injections treatment was started three days before ceding the diet and persisted 9 days. Injections contained 1% roflumilast (1 mg/kg or 3 mg/kg) dissolved in DMSO (VWR prolabo, Australia) and diluted in 2% Tween80 in 0.5% methyl cellulose. Injections were given twice a day with a volume of 5 μl/gram mouse. At the end of the treatment period, all remaining animals were sacrificed by an intraperitoneal injection of sodium dolethal (200 mg/kg) followed by transcardial perfusion (10 U/ml heparin in 1×PBS). One animal died for unknown reasons.

Gebr32a Study

At the start of the experiment, all animals were phenotyped for baseline cognitive performance in the object location task. Afterwards, 4 groups were defined (n1=34, n2=34, n3=22, n4=22) and group 2, 3 and 4 were subjected to a 0.3% w/w cuprizone diet (Bis(cyclohexanone)-oxaldihydrazone) (Sigma-Aldrich, United States) for 42 days. All groups were phenotyped for cognitive performance at the end of the demyelination phase, preceding treatment. For intermediate post mortem analysis, 12 animals of group 1 (no cuprizone) and 12 animals of group 2 (cuprizone) were sacrificed by an intraperitoneal injection of sodium dolethal (200 mg/kg) followed by transcardial perfusion (10 U/ml heparin in 1×PBS). Subcutaneous injections treatment was started three days before ceding the diet and persisted 9 days. Injections contained 1% Gebr32a (0.1 mg/kg or 0.3 mg/kg) dissolved in DMSO (VWR prolabo, Australia) and diluted in 2% Tween80 in 0.5% methyl cellulose. Injections were given twice a day with a volume of 5 μl/gram mouse. At the end of the treatment period, all remaining animals were sacrificed by an intraperitoneal injection of sodium dolethal (200 mg/kg) followed by transcardial perfusion (10 U/ml heparin in 1×PBS). One animal died for unknown reasons.

Object Location Task

The behavioral tasks were conducted in a dimly lighted room during the tests (19 lux). The room was designed symmetrically and the arena belonging to each test was placed right beneath the vent to avoid bias. The animals were randomly subjected to the different test. All experiments were performed blinded.

A transparent circular arena, made of polyvinyl chloride and with a diameter of 40 cm, was half-covered with white paper for the object location task. Two identical objects (four sets of object) were placed inside the arena according to the separation line between the covered and transparent wall. The available objects were: (1) a transparent glass bottle (diameter 2.7 cm, height 8.5 cm) filled with sand and water, (2) a massive metal cube (2.5 cm×5 cm×7.5 cm) with two holes (diameter 1.5 cm), (3) a cone made of brass (maximal diameter 6 cm and total height 3.8 cm), and (4) a massive aluminum cube with a tapering top (4.5 cm×4.5 cm×8.5 cm). The objects were offered to the animals according to a randomized scheme to avoid object nor place bias due to preferences. Before each trial, animals were placed in an empty incubation cage to increase the animal's interest. Within the first learning trial (T1), the objects were place symmetrically inside the arena and the animal was allowed to explore the objects and the arena for four minutes. Afterwards, the animal was placed back inside his home cage. After a predefined interval time (e.g. 3 h), the animals were placed in the incubation cage once again before entering the arena for trial 2 (T2) in which one of the two objects was moved. The time spent exploring each object was recorded manually using a computerized program. Exploration was defined as touching the object with the nose, except when the animal was sitting on the object. Between each trial, the objects, the arena, as well as the incubation cage was cleaned with 70% ethanol to avoid olfactory bias. Based on the calculations of the discrimination ratio (d2), the results were analyzed. The d2 value is defined as: (the time spent exploring the moved object—the time spent exploring the stationary object)/total exploration time in T2. The resulting value ranges between −1 and 1, in accordance to the level of discrimination towards the moved object. Mice were trained and tested at baseline for spatial memory performances in which they all performed significantly better than the hypothetical chance level of 0,0. Animals that not reached a total exploration time of 5 seconds were excluded from further analyses.

Transmission Electron Microscopy

The sample preparation for TEM was performed as described in Maheshwari et al. (2013) with minor modifications. Briefly, mice were transcardially perfused with lactated Ringer's solution under deep anaesthesia. A coronal brain block (1 mm thick) within the anteroposterior coordinates from −0.3 to −1.5 mm was cut in the midsagittal plane. Next, tissue was fixed with 2% glutaraldehyde and post fixated with 2% osmiumtetroxide in 0.05M sodium cacodylate buffer (pH=7.3) for 1 h at 4° C. The tissue was then stained with 2% uranyl acetate in 10% acetone for 20 min, dehydrated through graded concentrations of acetone and embedded in epoxy resin (araldite). Semithin sections (0.5 μm) were stained with a solution of thionin and methylene blue (0.1% aqueous solution) for light microscopic examination to delineate the region of interest. Subsequently, ultrathin sections (0.06 μm) were cut and mounted on 0.7% formvar-coated grids and contrasted with uranyl-acetate followed by lead citrate and examined on a Philips EM 208 transmission electron microscope (Philips, Eindhoven, The Netherlands) operated at 80 kV. Quantification was done using Fiji ImageJ by defining the G-ratio (diameter of the axon/diameter of the axons including the myelin sheath) of 100 axon. Axons that not reached a G-ratio of 0.968 (‘bare axons’) were excluded from further analysis.

RNA Isolation, cDNA Synthesis and Quantitative PCR

Total RNA was prepared using the RNeasy mini kit (Qiagen, Venlo, The Netherlands) according to the manufacturer's instructions with the following modification: Qiazol lysis reagent (Qiagen was used as lysis buffer. RNA concentration and purity was determined using a Nanodrop spectrophotometer (Isogen Life science). Consequently, cDNA synthesis was performed using the qScript cDNA SuperMix (Quanta Biosciences, Boston, USA). Quantitative PCR was conducted on a StepOnePlus™ Real-Time PCR system (Applied biosystems, Ghent, Belgium). The SYBR green master mix (Applied biosystems), 10 μM of forward and reverse primers, nuclease free water and 12.5 ng template cDNA in a total reaction volume of 10 μl. Relative quantification of gene expression was accomplished by using the comparative Ct method. Data were normalized to the most stable reference genes.

Oligodendrocyte Precursor Cell Isolation

Mixed glial cultures were prepared from postnatal day 2 mouse cerebral cortices of C57BLl/6JOIaHsd animals and used to generate OPC-enriched glial cultures by separating the OPCs from the astrocyte monolayer by orbital shaking followed by purification by differential adhesion to plastic. Purified OPCs were seeded on poly-L-lysine (5 μg/ml; Sigma-Aldrich, Bornem, Belgium) coated plates or glass cover slides for staining. Isolated OPCs were plated in 24-well plates (150,000 cells/well Greiner Bio-One, Frickenhausen, Germany) for immunocytochemistry or in 6-well plates (500,000 cells/well Greiner Bio-One, Frickenhausen, Germany) for western blot analyses. OPCs were induced to differentiate for 6 days with Roflumilast (5 μM and 10 μM), Gebr32 (0.5, 1 and 5 μM) or vehicle (DMSO) in SATO-medium supplemented with 2% horse serum (Sigma-Aldrich). Treatment was repeated on day 2 and 4, applying a 40% medium change. All plates were at 37° C. and 8.5% C02.

Western Blot

Total protein extraction was performed by homogenizing the samples in radioimmunoprecipitation assay (RIPA) buffer (150 mM sodium chloride, 1.0% Triton X-100, 0.5% sodium deoxycholate, 0.1% SDS, 50 mM Tris, pH 8.0) containing an EDTA, protease (complete Ultra tablets, Mini Easypack, Roche) and phosphatese (PhosSTOP EASYpack, Roche) inhibitor cocktail. Protein samples were centrifuged at 12 000×g for 15 min at 4° C. Total protein concentration was assess using a Pierce™ BOA Protein Assay Kit (Thermo Fisher Scientific) according to the manufacturer's instruction.

Western blot was used to assess the total MBP production by OPC treated with roflumilast or Gebr32a compare to the vehicle group. Equal amount of protein sample was separated by 10% sodium dodecyl sulfate polyacrylamide gel electrophorese and blotted onto a PVDF membrane (GE Healthcare, Buckinghamshore, UK). A visualization step was performed to assess protein separation and to check transfer efficiency by staining the membrane with Ponceau red. The membrane was transferred into a blocking buffer (4% non-fat dry milk, Tris-buffered saline with 0.1% Tween-20) for 1 hour at room temperature. Primary antibodies were incubated: rat anti-MBP (1/500, MAB386 Millipore) and Mouse Anti-β-actin (1/1000, Santa Cruz Biotechnology) for 2 hours at room temperature. After washing with TBS-T (Tris-buffered saline with 0.1% Tween-20) membranes were incubated with secondary antibodies: horseradish peroxidase-conjugated rabbit-anti mouse and goat anti-rat antibodies (Dako, 1:2000) for 1 hour at room temperature. An ECL Plus detection kit (Thermo Fisher Scientific) was used and the generated chemiluminescent signal was detected by a luminescent image analyzer (ImageQuant LAS 4000 mini; GE Healthcare).

Organotypic Cerebellar Brain Slice

Wild-type C57BI/6J OlaHsd pups (P10) were used to prepare organtoypic cerebellar slices. Cerebella was extracted and meninges was dissected in 0.1% PBS/glucose. The tissue was cut into 270 μm thick saggital slices of the medial cerebellum with a tissue chopper. Slices were transferred to 0.1% PBS/glucose dissection medium, separated and plated on 24-well plate Millipore-Millicel-CM culture inserts (Fisher Scientifice, Canada) with 2 slices per insert. Culture media was composed of 50% minimal essential media (MEM) (Gibco), 25% Earle's balanced salt solution (EBSS) (Gibco), 25% heat-inactivated horse serum, 1% Penicillin/Streptomycin, 1% GlutaMax (200 nM) and 6.5 mg/ml glucose. Every 2 to 3 days, 60% of the medium was replaced with fresh media. Demyelination was induced after 6 days in culture with incubation of 0.5 mg/ml lysolecithin for 16 h. Next, membranes were allowed to recover for 24 h in fresh culture medium. Treatment was started afterwards (5 μM Roflumilast, 0.5 μM Gebr32a) and continued for 14 days.

Immunohistochemistry

MBP and O4 staining of OPC OPCs were fixed in 4% paraformaldehyde. Next, cells were incubated with blocking buffer (5% bovine serum albumin (BSA) and 0.05% Tween 20 in PBS) for 30 min at room temperature. Primary antibodies were incubated: rat anti-MBP (1/500, MAB386 Millipore) and Mouse Anti-O4 (1/750, MAB1326 R&D systems) for 4 hours at room temperature and washed three times with PBS. The glass cover slides with OPCs were incubated for 1 hour in the dark at room temperature with secondary conjugated antibodies: goat anti-rat coupled to Alexa555 (1/600 in PBS, molecular probes) and goat anti-mouse IgM coupled to Alexa488 (1/600 in PBS, molecular probes). Nuclear staining was performed using 4,6′-diamidino-2-phenylindole (DAPI; Invitrogen) for 10 minutes. The glass cover slides with OPC's were mounted with DAKO fluorescence mounting medium. Fluorescence analysis was performed using the Nikon eclipse 80i microscope and NIS Elements BR 3.10 software (Nikon, Japan). Quantification was done using Fiji ImageJ by defining the ratio of MBP/O4+ cells and defining the pixel intensity of the MBP staining.

Post Mortem Brain Section Staining

Isolated brain tissues were fixed in 4% paraformaldehyde overnight and cryoprotected with sucrose gradient. Next, tissue was sectioned at 10 μm and stained for MBP. Briefly, sectioned were air-dried and fixed in acetone for 10 minutes. Non-specific binding was blocked using 10% DAKO protein block in PBS for 30 minutes. Sections of each tissue sample were incubated with rat anti-MBP (1/500, MAB386 Millipore) for 1 h at room temperature followed by incubation with Alexa-555-labeled goat anti-rat (1/600 in PBS, Molecular probes). Analysis was carried out using a Nikon eclipse 80i microscope and NIS Elements BR 4.20 software (Nikon). Quantification was using Fiji ImageJ done by assessing the thickness of the corpus callosum corrected for the degree of myelination defined by the pixel intensity. Due to low quality of the tissue and difficulties in preparing 10 μm thick slices, brain sections of 4 animals could not be quantified.

Organotypic Cerebellar Brain Slice Staining

Organotypic cerebellar brain slices were fixed in 4% paraformaldehyde at room temperature for 40 minutes and incubated with primary antibodies diluted in blocking buffer (5% bovine serum albumin (BSA) and 0.05% Tween 20 in PBS) for 4 h at room temperature. The used antibodies included rat anti-MBP (1/500, MAB386 Millipore) and rabbit anti-neurofilament (1/750, N4142 Sigma Aldrich). Next, slices were incubated with Alexa-488-labeled goat anti-rabbit (1/600 in PBS, Molecular probes) and Alexa-555-labeled goat anti-rat (1/600 in PBS, Molecular probes) for 1 h at room temperature. Nuclear staining was performed using 4,6′-diamidino-2-phenylindole (DAPI; Invitrogen) for 10 minutes and insert membranes were transported onto mounting glasses. Analysis was carried out using a Nikon eclipse 80i microscope and NIS Elements BR 4.20 software (Nikon). Quantification was done using Fiji ImageJ by counting the percentage of myelinated axons crossing a predefined cross-sectional path.

Statistical Analysis

For evaluation of the human PDE4D splice variant expression, a two side paired (OLg and OPC) or non-paired (brain lesion) student t-test was performed. Immunocytochemical staining were quantified using a non-parametric Kruskal-Wallis test with Dunn's multiple comparisons. Western blot data were analysed using a non-parametric Mann-Withney test. For the object location task, a one sample t-test was used to compare the average value results of the animals to the chance level of 0.0. Differences between groups in cognitive performances and post mortem analyses was assessed using a one-way ANOVA with Tukey's multiple comparison test. Data were statistically analyzed with GraphPad Prism 6 for windows and are reported as mean values ±standard error (SEM). Outliers were determined by Dixon's principles of exclusion of extreme values [36]. *P<0.05, **P<0.01, ***P<0.001

Experimental Autoimmune Encephalomyelitis

Ten-weeks-old female C57bl6 mice (n=56) were immunized in the flank and neck with 200 μg/mouse MOG35-55 peptide (Hooke) emulsified in Complete Freund's Adjuvant containing Mycobacterium tuberculosis. Immediately after immunization, treatment was started and lasted for 27 days. Treatment consisted of twice a day a s.c. injection with a 1% DMSO in 0.5% methylcellulose solution, roflumilast (0.3 mg/kg or 3 mg/kg in vehicle) or Gebr32a (0.3 mg/kg in vehicle). All animals were sacrificed at day 27. Animals were neurologically scored for clinical signs on a daily basis using the following scores: 0=normal behavior, 0.5=distal limp tail, 1=complete limp tail, 2=limp tail and hind leg inhibition, 2.5=limp tail and weakness of hind legs, 3=limp tail and dragging of hind legs, 3.5=limp tail, complete paralysis of hind legs and animal is unable to right itself when placed on its side, 4=limp tail, complete hind leg paralysis and partial front leg paralysis, 4.5=limp tail, complete hind leg paralysis, partial front leg paralysis and no movement around the cage, 5=death.

A two-way ANOVA with Tukey's multiple comparison test was performed to test for statistical differences.

Differentiation of Primary Rat Schwann Cells

Primary rat Schwann cells were isolated from p3 Wistar rats. Primary rat Schwann cells were cultured onto an aligned network of electrospinned fibers and stimulated with vehicle (0.1% DMSO), the pan-PDE4 inhibitor Roflumilast (5 μM or 10 μM) or the selective PDE4D inhibitor BPN14770 (1 μM or 5 μM), both dissolved in 0.1% DMSO. Cells were fixated at day 14 and stained for MBP (red) and MAG (green), both markers for Schwann cells differentiation. MBP and β-actin protein expression was analyzed using western blot analysis on primary rat Schwann cells (500.000 cells). For gene expression analyses, the primary rat Schwann cells were lysed after 6 days and RNA was subsequently isolated. qPCR was conducted to evaluate changes in mRNA expression. Data (n≥8/group) are displayed as mean+/−SEM. Data were analyzed using a one-way ANOVA with Dunnett's multiple comparison test (compared to control); *p<0.05, **p<0.01, ***p<0.001. qPCR was conducted to evaluate changes in mRNA expression of MBP, PLP, MAG and SOX10. Data (n≥8/group are displayed as mean+/−SEM. Data were analyzed using a one-way ANOVA with Dunnett's multiple comparison test (*p<0.05, **p<0.01, ***p<0.001 compared to control conditions)

### Example 1: PDE4D Inhibition Stimulates OPC Differentiation In Vitro

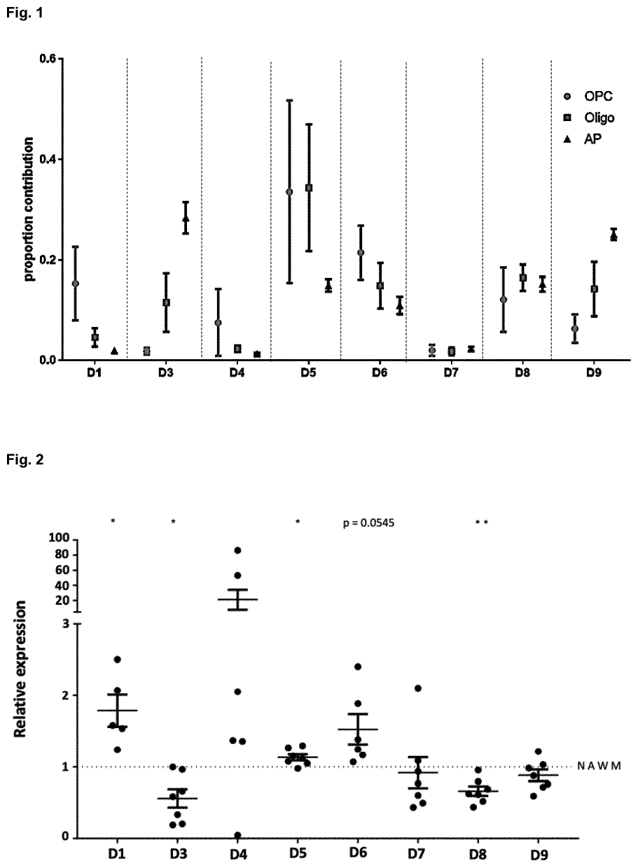

Expression levels of different PDE4D splice variants (PDE4D1, PDE4D3, PDE4D7, PDE4D8, PDE4D9) differ depending on human oligodendrocytes (OLG) and oligodendrocyte precursor cells (OPC) (FIG. 1). Furthermore, it was shown that expression of PDE4D1 was significantly lower in normal appearing white matter (NAWM) than in MRI-confirmed chronic inactive multiple sclerosis lesions (FIG. 2).

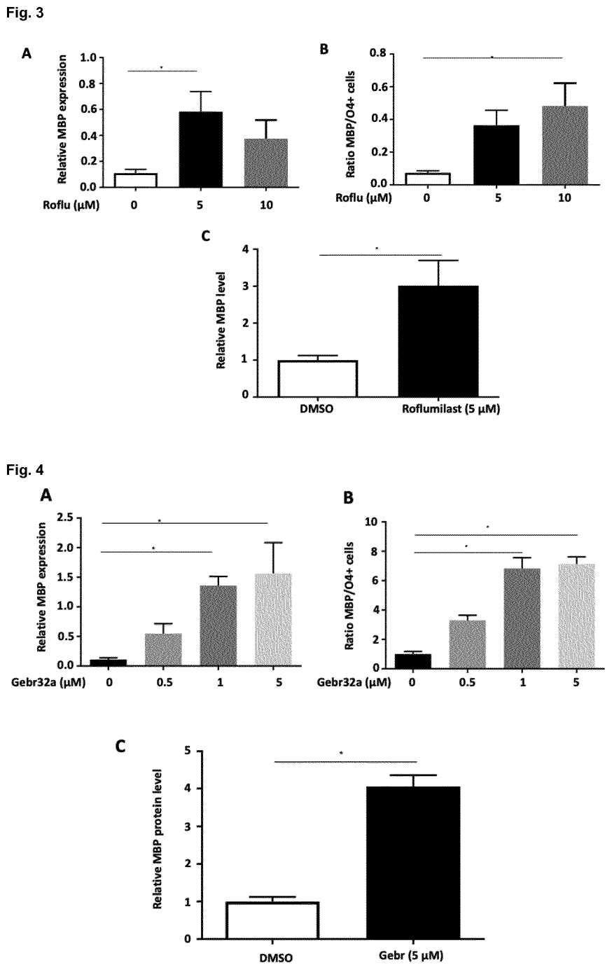

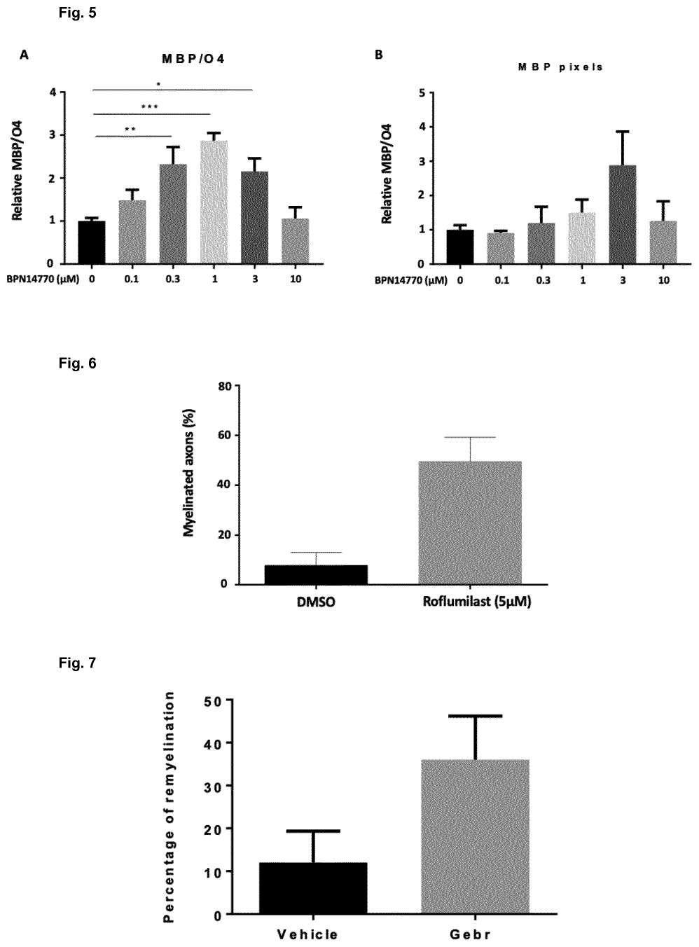

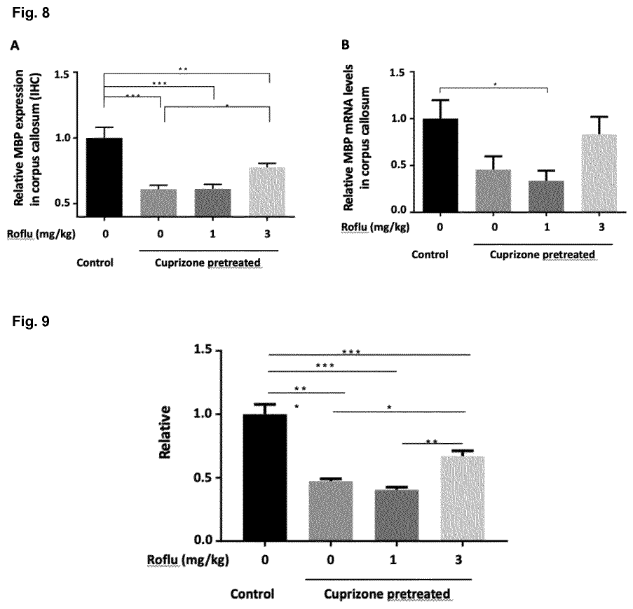

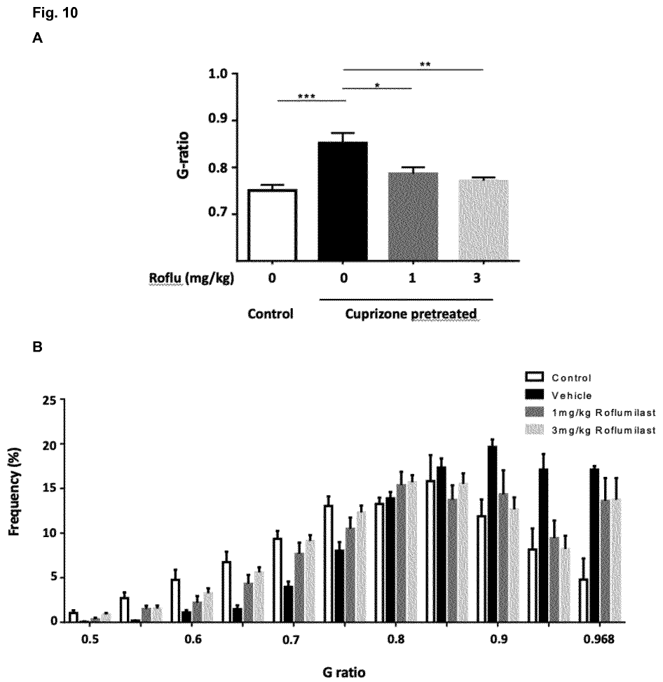

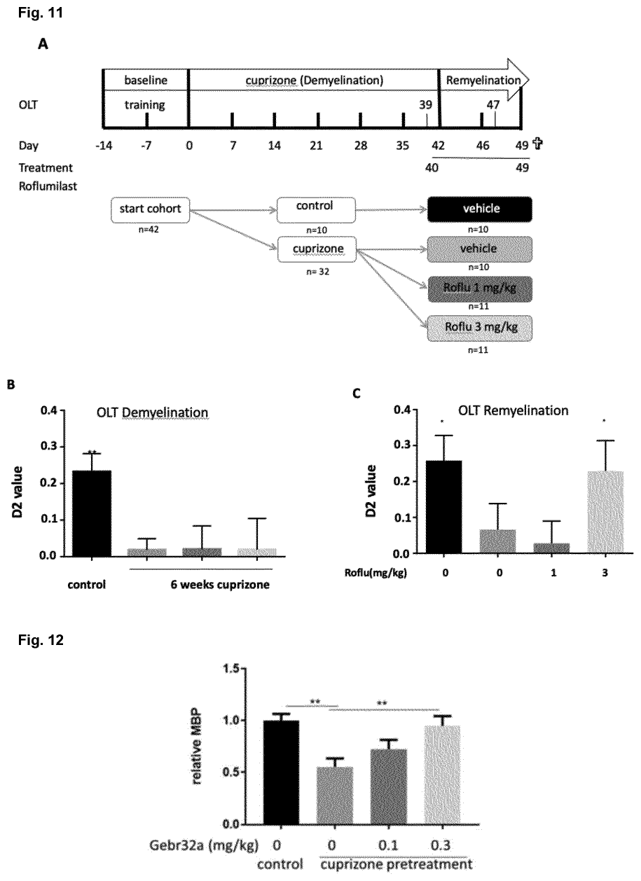

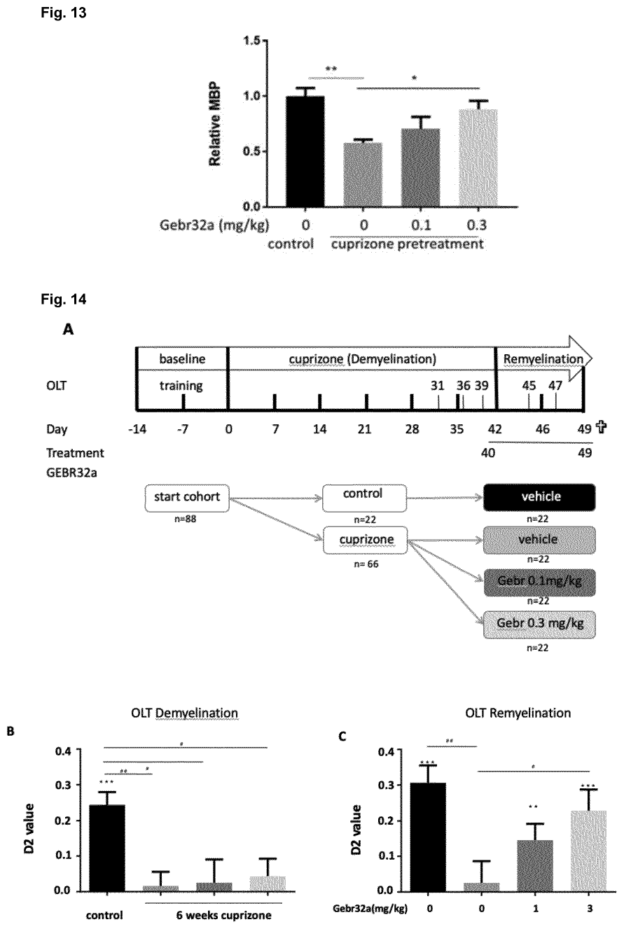

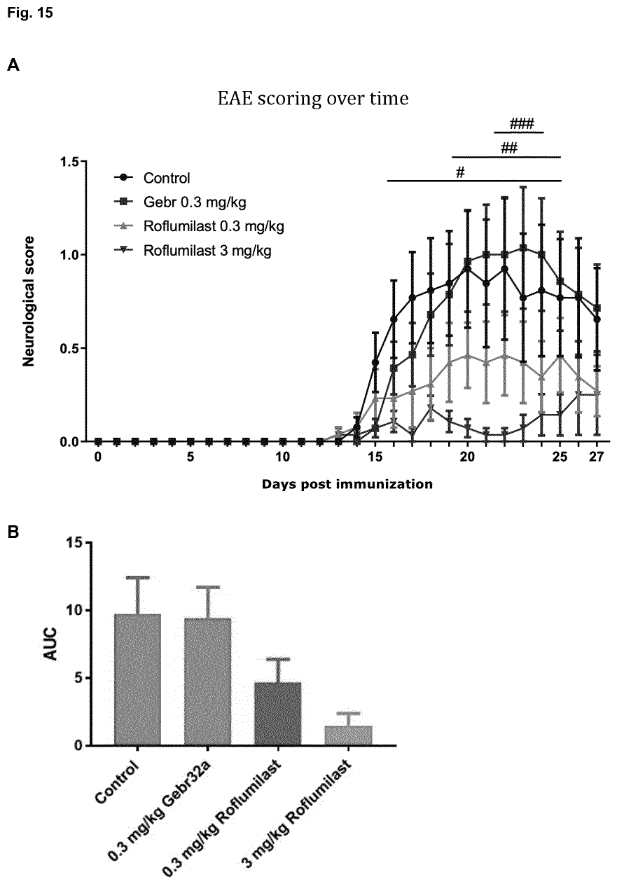

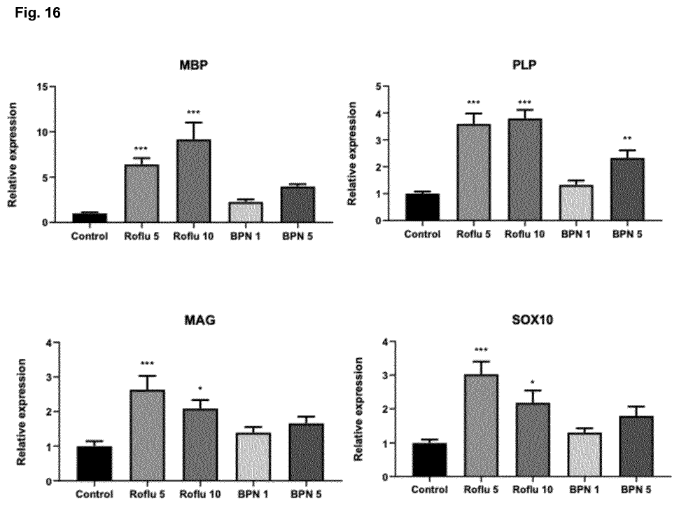

Differentiation of OPC's into MBP-expressing oligodendrocytes is a prerequisite for (re)myelination. The full (or pan-) PDE4 inhibitor roflumilast, which inhibits all of the PDE4 isoforms, including the above-mentioned PDE4D splice variants, induced primary mouse OPCs to differentiate into mature oligodendrocytes. Relative MBP protein expression was three-fold increased by 5 μM roflumilast compared to vehicle in western blot (P<0.05) and IHC (P<0.05) (FIG. 3). To investigate whether specific PDE4D inhibition was sufficient to induce OPC differentiation, the specific PDE4D inhibitors GEBR32a and BPN14770 were tested for their potency to induce OPC differentiation. Both PDE4D inhibitors induced in vitro OPC differentiation six days after the treatment. GEBR32a treatment yielded a dose-dependent increase in MBP protein expression and MBP/O4 ratio (1 μM:P<0.05 and 5 μM:P<0.05; FIG. 4). The induction of MPB protein expression by 5 μM GEBR32a was confirmed by western blot analyses, displaying a three-fold increase in MPB expression (P<0.05). BPN14770, another PDE4D inhibitor, confirmed target specify by inducing MBP/O4 ratio (0.3 μM:P<0.01; 1 μM:P<0.001; 3 μM P<0.05) (FIG. 5). Thus, specific PDE4D inhibition induced MBP expression reflective of the induction of in vitro OPC differentiation.

### Example 2: PDE4D Inhibition Accelerates Ex Vivo (Re)Myelination in Lysolecithin-Demyelinated Brain Slices

Lysolecithin-demyelinated ex vivo cerebellar brain slices of 10-days old mice pups were treated with roflumilast, GEBR32a, or vehicle. A 14-days treatment with roflumilast (5 μM) and GEBR32a (0.5 μM) resulted in a four-fold increase in MBP alignment with neurofilament, a neuronal marker (FIGS. 6 and 7). Alignment of MBP to neurofilament are hallmarks of (re)myelinated nude axons. Specific PDE4D inhibition improved ex vivo remyelination.

### Example 3: PDE4D Inhibition Improves In Vivo Remyelination in Cuprizone-Demyelinated Brain Regions

To investigate whether PDE4 inhibition improved in vivo remyelination, vehicle or two dosages roflumilast (1 mg/kg and 3 mg/kg) were administered to cuprizone demyelinated mice (six weeks; 0.3% w/w) and compared a non-demyelinated vehicle-treated control group (FIG. 11a). IHC staining for MBP expression in the corpus callosum and hippocampus (e.g. dendate gyrus) was quantified as readout for remyelination. A one-way ANOVA with Tukey post-hoc analysis, revealed that the 3 mg/kg roflumilast-treated animals showed an increased MBP expression in the corpus callosum (P<0.05) and dendate gyrus (P<0.05), 9 days after the start of the treatment compared to the vehicle-treated cuprizone-demyelinated mice (FIGS. 8 and 10). In line, ultrastructural TEM analyses were performed in the corpus callosum to determine the G ratio, e.g. the ratio of the inner axonal diameter to the total outer diameter representing myelination of axons; a higher G ratio represents demyelination. A one-way ANOVA with Tukey's multiple comparison test confirmed that 1 mg/kg (P<0.05) and 3 mg/kg (P<0.01) roflumilast treatment yielded a re-establishment of the G ratio to control levels, featuring remyelination (FIG. 7). Relative mbp gene expression was not altered in the corpus callosum (FIG. 8). In line, we examined whether roflumilast treatment could reverse the hippocampus-dependent demyelination-induced functional deficits in spatial memory in the object location task. Cognitive performance (e.g. spatial memory in the object location task) was used as functional readout for remyelination. The 3 mg/kg roflumilast-treated group showed a significant recovery of spatial memory to a level of the control animals (one-sample t-test P<0.05). In contrast, the vehicle and 1 mg/kg roflumilast treated cuprizone animals did not show a recovery of the spatial memory (FIG. 11).

To determine whether narrowing down the target specificity to PDE4D inhibition was sufficient to induce in vivo remyelination, we administered the specific PDE4D inhibitor GEBR32a in the cuprizone model. A one-way ANOVA with Tukey post-hoc analysis, revealed that 0.3 mg/kg GEBR32a treatment significantly increased MBP expression in the corpus callosum (P<0.01; FIG. 12) and dendate gyrus (P<0.05), 9 days after the start of the treatment compared to the vehicle treated mice. In contrast, the 0.1 mg/kg GEBR32a treatment was not sufficient to significantly improve MPB expression in the corpus callosum or dendate gyrus. Moreover, GEBR32a-treated groups showed a significant dose-dependent functional recovery of spatial memory, related to remyelination (FIG. 14). GEBR32a treatments restored the spatial discrimination index (d2) to a level comparable to that of the control animals and significantly improved d2 compared to vehicle-treated cuprizone-demyelinated mice (0.1 mg/kg, P<0.05; 0.3 mg/kg P<0.01; FIG. 13). Compared to roflumilast treatment, we showed that GEBR32a treatment induces remyelination at a 10-fold lower dosage. We conclude that narrowing down the target specificity to PDE4D inhibition is an efficient manner to induce remyelination in animal model for demyelination.

### Example 4: Experimental Autoimmune Encephalomyelitis (EAE)

Prophylactic treatment with Gebr32a (0.3 mg/kg) has no effect on the clinical score of the mice with EAE, whereas roflumilast treated animals (0.3 mg/kg and 3 mg/kg) showed a dose-dependent decrease in disease score (FIG. 15 A-B).

Thus, the specific PDE4D inhibitor Gebr32a did not improve the disease course in the inflammatory experimental autoimmune encephalomyelitis model (EAE) using the repair inducing dose of Gebr32a (0.3 mg/kg). In contrast, animals treated with the pan-PDE4 inhibitor roflumilast (0.3 mg/kg and 3 mg/kg) showed a dose-dependent attenuation of the disease score.

### Example 5: Differentiation of Primary Rat Schwann Cells

Inhibition of PDE4 by roflumilast and inhibition of the specific isoform PDE4D by BPN14770 induced differentiation of primary rat Schwann cells. In particular, immunohistochemical staining for MBP and MAG, two markers of Schwann cell differentiation, was performed on primary rat Swann cells cultured in the presence of Roflumilast or BPN14770. IHC staining showed that MBP and MAG protein expression increases dose-dependently upon both PDE4 or PDE4D inhibition (data not shown). These data were confirmed using western blot analysis (not shown). Furthermore, qPCR analysis for the differentiation markers MBP, MAG, PLP and SOX10 showed that the expression of all these genes was increased upon PD4E or PDE4D inhibition (FIG. 16).

### REFERENCES

Zhang, Y., et al., An RNA-sequencing transcriptome and splicing database of glia, neurons, and vascular cells of the cerebral cortex. J Neurosci, 2014. 34(36): p. 11929-47.
Bruno, O., et al., GEBR-7b, a novel PDE4D selective inhibitor that improves memory in rodents at non-emetic doses. Br J Pharmacol, 2011. 164(8): p. 2054-63.
Giembycz, M. A., 4D or not 4D—the emetogenic basis of PDE4 inhibitors uncovered? Trends Pharmacol Sci, 2002. 23(12): p. 548.
Maheshwari, A., et al., Local overexpression of interleukin-11 in the central nervous system limits demyelination and enhances remyelination. Mediators Inflamm, 2013. 2013: p. 685317.
Li Z., et al., Identification of novel phosphodiesterase-4D inhibitors prescreened by molecular dynamics-augmented modelling and validated by bioassay. Journal of Chemical Information and Modeling, 2013. 53: p 972-981.
Sierksma, A. S., et al., Improvement of spatial memory function in APPswe/PS1dE9 mice after chronic inhibition of phosphodiesterase type 4D. Neuropharm., 2014. 77: 120-130.

1-22. (canceled)

23. A method for diagnosing, preventing, and/or treating a demyelinating disease of the nervous system in a subject, the method comprising:
administering to the subject a selective PDE4D inhibitor that selectively inhibits type D isoforms of PDE4.

24. The method of claim 23, wherein the selective PDE4D inhibitor inhibits maximum 45% of the activity of the type A, B, and C isoforms of PDE4.

25. The method of claim 23, wherein the selective PD4D inhibitor inhibits at least 50% of the activity of the type D isoforms of PDE4.

26. The method of claim 23, wherein the selective PD4D inhibitor restores the remyelination process in the treatment of the demyelinating disease in the subject.

27. The method of claim 23, wherein the demyelinating disease is a demyelinating disease of the central nervous system.

28. The method of claim 27, wherein the demyelinating disease is multiple sclerosis.

29. The method of claim 28, wherein the multiple sclerosis is progressive multiple sclerosis.

30. The method of claim 29, wherein the progressive multiple sclerosis is selected from the group comprising primary progressive multiple sclerosis, secondary progressive multiple sclerosis, and relapse remitting multiple sclerosis.

31. The method of claim 23, wherein the demyelinating disease is a demyelinating disease of the peripheral nervous system.

32. The method of claim 31, wherein the demyelinating disease is selected from the group consisting of diabetic neuropathy, Marie-Charcot tooth disease, and traumatic nerve injury.

33. The method of claim 23, wherein the subject is a non-human animal or a human.

34. The method of claim 23, wherein the selective PDE4D inhibitor is a compound of formula (I) or a pharmaceutically acceptable salt thereof: 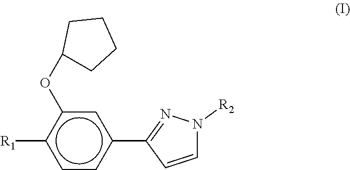
where:
R₁ and R₂ are independently selected from the group consisting of —OH, —NH₂, halo, —C₁₋₈ alkyl, and C₁₋₈ alkoxy-, wherein said —C₁₋₈ alkyl and C₁₋₈ alkoxy- are optionally substituted with one or more groups selected from the group consisting of —OH, —NH₂, halo, Ar₁, and Het₁;
Ar₁ represents a polyunsaturated aromatic hydrocarbyl group having a single ring or multiple aromatic rings fused together or linked covalently and containing 6 to 10 atoms, wherein at least one of the single ring or multiple aromatic rings is aromatic; and
Het₁ represents a morpholino ring or a 5 to 12 carbon-atom aromatic ring or ring system containing 1 to 3 rings that are fused together or linked covalently, wherein each of the 1 to 3 rings contains 5 to 8 atoms, wherein at least one of the 1 to 3 rings is aromatic, and wherein one or more carbon atoms in any of the 1 to 3 rings optionally is replaced by an oxygen atom, a nitrogen atom, or a sulfur atom.

35. The method of claim 34, wherein:
R₁ is a C₁₋₈alkoxy- optionally substituted with one or more groups selected from —OH, —NH₂, and halo;
R₂ is a —C₁₋₈alkyl optionally substituted with one or more groups selected from —OH and Het₁; and
Het₁ represents a morpholino ring or a 5 to 6 carbon-atom aromatic ring, wherein one or more carbon atoms of the morpholino ring or the aromatic ring is optionally replaced by an oxygen atom, a nitrogen atom, or a sulfur atom.

36. The method of claim 34, wherein:
R₁ is a C₁₋₈alkoxy- optionally substituted with one or more groups selected from halo;
R₂ is a —C₁₋₈alkyl optionally substituted with one or more groups selected from —OH and Het₁; and
Het₁ represents a morpholino ring or a 5 to 6 carbon-atom aromatic ring, wherein one or more carbon atoms of the morpholino ring or the aromatic ring is optionally replaced by an oxygen atom or a nitrogen atom, or a sulfur atom.

37. The method of claim 34, wherein:
R₁ is difluoromethoxy;
R₂ is a —C₁₋₈alkyl optionally substituted with one or more groups selected from —OH and Het₁; and
Het₁ represents a morpholino ring.

38. The method of claim 23, wherein the selective PDE4D inhibitor is a compound of formula (II) or a pharmaceutically acceptable salt thereof: 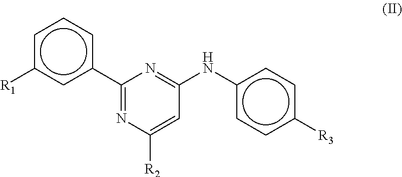
where:
R₁, R₂, and R₃ are independently selected from the group consisting of —OH, —NH₂, halo, —C₁₋₈ alkyl, C₁₋₈ alkoxy-, and —C₁₋₈ alkylamine, wherein the —C₁₋₈ alkyl, the C₁₋₈ alkoxy-, and the —C₁₋₈alkylamine are optionally substituted with one or more groups selected from —OH, -ME, halo, oxo, Ar₁, and Het₁;
Ar₁ represents a polyunsaturated aromatic hydrocarbyl group having a single ring or multiple aromatic rings fused together or linked covalently and containing 6 to 10 atoms, wherein at least one of the single ring or multiple aromatic rings is aromatic; and
Het₁ represents a 5 to 12 carbon-atom aromatic ring or ring system containing 1 to 3 rings that are fused together or linked covalently and containing 5 to 8 atoms each, wherein at least one of the 1 to 3 rings is aromatic, and wherein one or more carbon atoms in one or more of the 1 to 3 rings optionally is replaced by an oxygen atom, a nitrogen atom, or a sulfur atom.

39. The method of claim 38, wherein:
R₁ is halo;
R₂ is a —C₁₋₈ alkyl optionally substituted with one or more halo; and
R₃ is a —C₁₋₈ alkylamine optionally substituted with one or more oxo.

40. The method of claim 23, wherein the selective PDE4D inhibitor is selected from the group consisting of 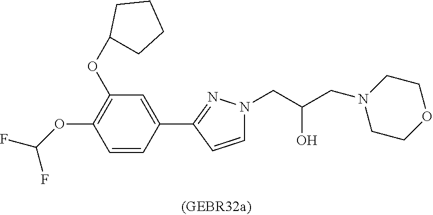 [SMILES: OC(CN1CCOCC1)Cn1ccc(-c2ccc(OC(F)F)c(OC3CCCC3)c2)n1]
or pharmaceutically acceptable salts thereof, and 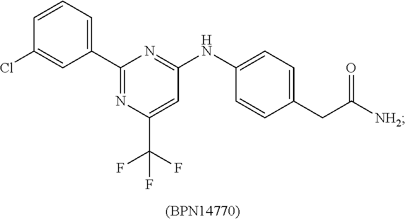 [SMILES: NC(=O)Cc1ccc(Nc2cc(C(F)(F)F)nc(-c3cccc(Cl)c3)n2)cc1]
or pharmaceutically acceptable salts thereof.

41. The method of claim 23, wherein the selective PDE4D inhibitor is administered at a daily dose rate from 0.01 mg to 1000 mg.

42. The method of claim 23, wherein the selective PDE4D inhibitor is included within a pharmaceutical composition comprising the selective PDE4D inhibitor or pharmaceutically acceptable salt thereof in combination with at least one pharmaceutically acceptable carrier, diluent, excipient, and/or adjuvant and optionally one or more additional pharmaceutically active compound.
